(19) United States
(12) Patent Application Publication
(10) Pub. No.: US 2020/0215227 A1
(43) Pub. Date: Jul. 9, 2020

(54) BIOARTIFICIAL MEMBRANES HAVING CONTROLLED VISCOELASTICITY AND RIGIDITY FOR USE IN TISSUE ENGINEERING

(71) Applicants: UNIVERSIDAD DE GRANADA, Granada (ES); Servicio Andaluz de Salud, Sevilla (ES)

(72) Inventors: Víctor Sebastián CARRIEL ARAYA, Granada (ES); Fernando CAMPOS SÁNCHEZ, Granada (ES); Ricardo FERNÁNDEZ VALADÉS, Granada (ES); Modesto Torcuato LÓPEZ LÓPEZ, Granada (ES); María del Carmen SÁNCHEZ QUEVEDO, Granada (ES); Miguel ALAMINOS MINGORANCE, Granada (ES)

(21) Appl. No.: 16/341,592

(22) PCT Filed: Oct. 13, 2017
(86) PCT No.: PCT/ES2017/070680

§ 371 Date: Feb. 27, 2020

(30) Foreign Application Priority Data
Oct. 14, 2016 (ES) P201631327

(51) Int. Cl. A61L 27/22; A61L 27/20; A61L 27/38; A61L 27/54; A61L 27/02

(52) U.S. Cl. CPC A61L 27/225; A61L 27/20; A61L 2300/214; A61L 27/54; A61L 27/025; A61L 27/38

(57) ABSTRACT

The present invention belongs to the field of biomedicine and tissue engineering. Specifically, it relates to a biomaterial and to an in vitro method for preparing a tissue or bioartificial membrane having controlled elasticity and rigidity, and to the tissue or artificial membrane obtainable using the method. The invention further relates to the uses thereof in medicine.

The present invention belongs to the field of biomedicine and tissue engineering. Specifically, it relates to a biomaterial and to an in vitro method for preparing a tissue or bioartificial membrane having controlled elasticity and rigidity, and to the tissue or artificial membrane obtainable using the method. The invention further relates to the uses thereof in medicine. These tissues and membranes have the biological and chemical properties for use in tissue engineering (TE).

### PRIOR STATE OF THE ART

TE is an up-and-coming area in biomedical research which, through the use of cells, growth factors and biomaterials allows generating artificial tissues for restoring, replacing, or increasing the functional activities of organic tissues themselves. In this sense, biomaterials play a fundamental role since they provide the physicochemical and structural properties to tissues generated by TE. Furthermore, some biomaterials can promote certain cell functions such as, for example, proliferation, migration, and differentiation. Biomaterials used in TE can generally be of synthetic origin, a natural origin, and/or a combination of these components (Williams D F. Definitions in biomaterials: proceedings of a consensus conference of the European Society for Biomaterials, Chester, England, March 3-5. Elsevier. 1987), with natural biomaterials being more efficient and biocompatible than synthetic biomaterials.

At present, to be able to use a biomaterial in TE, a series of requirements must be met, where it is fundamental for such biomaterials to be biocompatible, non-toxic, chemically and mechanically stable, porous, and in some cases biodegradable or resorbable, and finally they must allow their structural and physical properties to be adapted according to specific needs (Cardona et al., 2011. Cornea. 30: 1428-1435).

One of the natural polymers most widely used in TE is fibrin, which is a biodegradable protein that participates in the natural tissue repair process after an injury. This biomaterial has been used to generate human skin, oral mucosa, and peripheral nerve substitutes (Meana et al., 1998. Burns 24: 621-630; San Martin et al., 2013. J Tissue Eng Regen Med. 2013; 7: 10-19; Kalbermatten et al., 2009. J Reconstr Microsurg. 25(1):27-33). Furthermore, fibrin served as the structural basis for generating fibrin-agarose (FA) hydrogels with which cornea, skin, oral mucosa, peripheral nerve, bladder, and cartilage models have been developed (Alaminos et al., 2006 Invest Ophthalmol Vis Sci. 47: 3311-3317; Carriel et al., 2012. Epithelial and stromal developmental patterns in a novel substitute of the human skin generated with fibrin-agarose biomaterials. Cells Tissues Organs, 196, 1-12; Rodriguez I A et al., 2012 J Tissue Eng Regen Med. 6: 636-644; Carriel et al., 2013. Combination of fibrin-agarose hydrogels and adipose-derived mesenchymal stem cells for peripheral nerve regeneration. J Neural Eng, 10, 026022). Although fibrin and FA have shown very positive results in various tissue models, their physical properties must be increased in order to extend their use in TE, especially in those cases where it is necessary to use more resistant biomaterials (bone tissue, abdominal wall repair, generation of biodegradable conduits, tendons, etc.).

Various (physical and chemical) techniques have been developed to date to improve the biomechanical properties of biomaterials, such as nanostructuring and cross-linking techniques. The nanostructuring technique, developed by the inventors of the present invention, demonstrated that it is possible to regulate structural characteristics, increase biomechanical properties, and conserve the biological properties of FA through a controlled compression and dehydration process (lonescu et al., 2011. J Mech Behav Biomed Mater 4: 1963-197; Scionti et al., 2014. J Biomed Mater Res. 102: 2573-25823). Conversely, chemical cross-linking techniques promote the formation of molecular interactions which entail increasing the biomechanical properties and structural changes of the biomaterials. In this sense, the most widely used chemical agents are aldehydes (glutaraldehyde, paraformaldehyde, formaldehyde), which promote covalent bonds (methylene bridges) between various proteins (Barnes et al., 2007. Tissue Eng. 13: 1593-1605; Cheng et al., 2013. Tissue Eng Part A. 19: 484-496). However, aldehydes are highly toxic, which limits their use in tissue engineering (Sung et al., 1999. J Biomater Sci Polym Ed. 10: 63-78; Mi et al., 2001. J Biomater Sci Polym Ed. 12: 835-850; Mi et al., 2002. Biomaterials 3: 181-191). Various chemical agents or cross-linkers, such as Genipin, which is extracted from the Gardenia jasminoide fruit (Yoo et al., 2011. Korean J Thorac cardiovasc. 44: 197-207) and has been used in traditional Chinese medicine and as a blue dye in food industries, have recently been described.

### BRIEF DESCRIPTION OF THE INVENTION

A first aspect of the invention relates to a biomaterial, hereinafter biomaterial of the invention, comprising:
a) fibrinogen (or fibrin);
b) an antifibrinolytic agent;
c) an element selected from: a coagulation factor, a source of calcium, thrombin, or any combinations thereof;
d) a polysaccharide; and
e) a compound of formula (I)

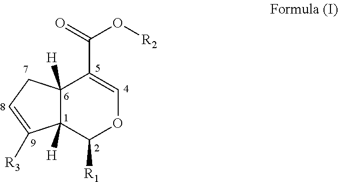

or any of the esters, tautomers, and pharmaceutically acceptable salts thereof, where:
   R₁ is —H, ═O or —OR₄, wherein R₄ is —H, C₁₋₆ alkyl, C₁₋₃ alkyl, or C₁₋₁₂ alkanoyl which can be substituted with phenyl, phenoxy, pyridyl, or thienyl;
   R₂ is H, C₁₋₆ alkyl, C₁₋₃ alkyl, methyl, ethyl, propyl, isopropyl, butyl, w-butyl, i-butyl, isobutyl, or sec-butyl;
   R₃ is a primary alcohol selected from —CH₂—OH and —R₅—CH₂—OH, where —R₅— is C₁₋₆ alkyl, C₁₋₃ alkyl, methyl, ethyl, propyl, isopropyl, butyl, n-butyl, t-butyl, isobutyl, sec-butyl.

A second aspect of the invention relates to an artificial tissue, hereinafter artificial tissue of the invention, comprising the biomaterial of the invention, and further comprising mammalian cells.

A third aspect relates to the use of the biomaterial or tissue of the invention in the production of a medicinal product, or alternatively, to the biomaterial or tissue of the invention for use in medicine.

A fourth aspect of the invention relates to the use of the biomaterial or tissue of the invention in the production of a medicinal product for increasing, restoring, or partially or completely substituting the functional activity of a diseased or damaged tissue or organ, or alternatively to the biomaterial or tissue of the invention for increasing, restoring, or partially or completely substituting the functional activity of a diseased or damaged tissue or organ.

A sixth aspect of the invention relates to a pharmaceutical composition comprising the artificial tissue of the invention.

A seventh aspect of the invention relates to a method for producing the artificial tissue of the invention comprising:
a) adding an antifibrinolytic agent to a composition comprising fibrinogen (or fibrin),
b) adding at least a coagulation factor, a source of calcium, thrombin, or any combination thereof to the product resulting from step (a);
c) adding a composition comprising a polysaccharide to the product resulting from step (b), and leaving to gel;
d) subjecting the product resulting from step (c) to a controlled nanostructuring process;
e) inducing the cross-linking of the resultant of step (b) and/or (d) with a compound of formula (I) as described in the first aspect of the invention;
f) seeding the products of step (c) and/or (e) with mammalian cells.

### DESCRIPTION OF THE FIGURES

FIG. 1. Typical dependence of shear stress according to shearing strain for the prepared hydrogels.

FIG. 2. Typical dependence of the viscoelastic modulus with strain amplitude for dynamic state measurements (sinusoidal oscillations with frequency equal to 1 Hz).

FIG. 3. Typical dependence of the viscoelastic modulus with strain frequency for dynamic state measurements (sinusoidal oscillations with amplitude frequency belonging to the linear viscoelastic region).

FIG. 4. Value of the modulus of rigidity of the prepared hydrogels (non-nanostructured -FAH (▪)- and nanostructured -NFAH (●)-) according to the concentration of the chemical agent Genipin.

FIG. 5. Value of the elastic modulus corresponding to the linear viscoelastic region (corresponding to a shearing strain of 0.01 and a frequency of 1 Hz) of the prepared hydrogels (non-nanostructured -FAH (▪)- and nanostructured -NFAH (●)-) according to the concentration of the chemical agent Genipin.

FIG. 6. Value of the viscous modulus corresponding to the linear viscoelastic region (corresponding to a shearing strain of 0.01 and a frequency of 1 Hz) of the prepared hydrogels (non-nanostructured -FAH (▪)- and nanostructured -NFAH (●)-) according to the concentration of the chemical agent Genipin.

FIG. 7. Scanning electron microscopy of fibrin-agarose hydrogels. FAH (m) shows the gels non-nanostructured, whereas NFAH (●) shows those hydrogels subjected to controlled nanostructuring. FA-CTR shows the constructs without cross-linking with Genipin, whereas GEN show the characteristic pattern of the hydrogels subjected to cross-linking with Genipin a 0.5 and 0.75%, respectively.

FIG. 8. Representative images of ex vivo histological analysis of the FA-CTR and CTR constructs subjected to cross-linking with Genipin.

FIG. 9. Morphological analysis of live-dead cell viability by fluorescence microscopy. This test shows viable and metabolically active cells in green, whereas dead cells are observed in red.

FIG. 11. Histological section of the skin of control Wistar rats, showing normal skin structure. 200 μm scale.

FIG. 12. Sample of the fibrin agarose hydrogel implantation area after 12 days. Picrosirius staining. 200 μm scale.

FIG. 13. Sample of the fibrin agarose hydrogel implantation area after 12 days. H2A staining. 50 μm scale.

FIG. 14. Sample of the fibrin agarose hydrogel implantation area after 26 days. H2A staining. 200 μm scale.

FIG. 15. Sample of the fibrin agarose hydrogel implantation area after 26 days. Picrosirius staining. 200 μm scale.

FIG. 16. Sample of the fibrin agarose hydrogel implantation area with 0.1% GP after 12 days. H2A staining. 200 μm scale.

FIG. 17. Sample of the fibrin agarose hydrogel implantation area with 0.1% GP after 12 days. Picrosirius staining. 50 μm scale.

FIG. 18. Sample of the fibrin agarose hydrogel implantation area with 0.1% GP after 26 days. H2A staining. 200 μm scale.

FIG. 19. Sample of the fibrin agarose hydrogel implantation area with 0.1% GP after 26 days. H2A staining. 50 μm scale.

FIG. 20. Sample of the fibrin agarose hydrogel implantation area with 0.25% GP after 12 days. H2A staining. 200 μm scale.

FIG. 21. Sample of the fibrin agarose hydrogel implantation area with 0.25% GP after 12 days. H2A staining. 50 μm scale.

FIG. 22. Sample of the fibrin agarose hydrogel implantation area with 0.25% GP after 26 days. H2A staining. 500 μm scale.

FIG. 23. Sample of the fibrin agarose hydrogel implantation area with 0.25% GP after 26 days. H2A staining. 50 μm scale.

### DETAILED DESCRIPTION

Genipin, or [Methyl (1R,2R,6S)-2-hydroxy-9-(hydroxymethyl)-3-oxabicyclo[4.3.0]nona-4,8-diene-5-carboxylate], is a chemical agent or cross-linker usually extracted from the Gardenia jasminoide fruit, characterized by presenting low cytotoxicity, and promoting an increase in the biomechanical properties of various matrices (Somers et al., 2008. J Heart Valve Dis. 17: 682-688; Chang et al., 2005. J Biotechnol. 120: 207-21933).

The authors of the present invention have designed a method for the synthesis of a biomaterial, which includes the addition of compounds of formula (I), and preferably Genipin, as a chemical agent for cross-linking, and the three-dimensional structure of which provides a support (scaffold) suitable for cell adherence, proliferation, and differentiation under suitable culture conditions.

Biomaterial of the Invention

A first aspect of the invention relates to a biomaterial, hereinafter biomaterial of the invention, comprising:

a) fibrinogen (or fibrin);

b) an antifibrinolytic agent;

c) an element selected from: a coagulation factor, a source of calcium, thrombin, or any combinations thereof;

d) a polysaccharide;

e) a compound of formula (I)

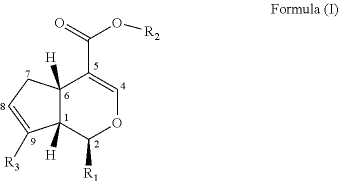

or any of the esters, tautomers, and pharmaceutically acceptable salts thereof, where:
R₁ is —H, ═O or —OR₄, wherein R₄ is —H, C₁₋₆ alkyl, C₁₋₃ alkyl, or C₁₋₁₂ alkanoyl which can be substituted with phenyl, phenoxy, pyridyl, or thienyl;
R₂ is H, C₁₋₆ alkyl, C₁₋₃ alkyl, methyl, ethyl, propyl, isopropyl, butyl, w-butyl, i-butyl, isobutyl, or sec-butyl;
R₃ is a primary alcohol selected from —CH₂—OH and —R₅—CH₂—OH, where —R₅— is C₁₋₆ alkyl, C₁₋₃ alkyl, methyl, ethyl, propyl, isopropyl, butyl, n-butyl, t-butyl, isobutyl, sec-butyl.

In another preferred embodiment, the biomaterial of the invention consists of:
a) Fibrinogen (or fibrin) as previously described,
b) an antifibrinolytic agent, preferably as previously described,
c) an element selected from: a coagulation factor, a source of calcium as previously described;
d) a polysaccharide as previously described,
e) a compound of formula (I) as previously described;

In this specification, “biomaterial” is understood as materials suitable for contacting the tissues of a subject for specific therapeutic, diagnostic, or preventive purposes, or as materials that can substitute a tissue. These materials must be biocompatible, i.e., must not cause any significant adverse effect on the living organism after the biomaterial interacts with bodily fluids and tissues, and, occasionally, must be biodegraded, either chemically or physically, or by a combination of both processes, to give rise to non-toxic components. The biomaterial according to the present invention comprises fibrinogen or fibrin, a polysaccharide, and a compound of formula (I), as previously defined.

Compound of Formula (I)

In a preferred embodiment, in the compound (I) of the biomaterial of the invention R₁ is —OR₄. In another preferred embodiment of this aspect of the invention, R₄ is —H or C₁₋₃ alkyl. In another preferred embodiment of this aspect of the invention, R₂ is H or C₁₋₃ alkyl and/or R₃ is —CH₂—OH, —CH₂—CH₂—OH, or —CH₂—CH₂—CH₂—OH. In another more preferred embodiment of this aspect of the invention, the compound used, Genipin, presents formula (II):

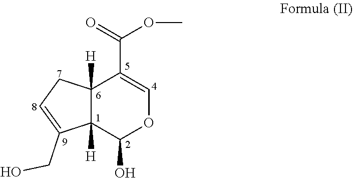 [SMILES: COC(=O)C1=CO[C@@H](O)[C@@H]2C(CO)=CC[C@H]12]

Fibrin

The formation of a fibrin matrix takes place by thrombin-induced polymerization of fibrinogen. Fibrinogen is a high molecular weight protein present in blood plasma. Thrombin is a proteolytic enzyme causing the cleavage of the fibrinogen molecule into low molecular weight polypeptides and into fibrin monomers. Said monomers polymerize into dimers and then bind to one another by means of covalent bonds by the action of factor XIII, previously activated by the thrombin, and in the presence of calcium ions.

In another preferred embodiment of this aspect of the invention, the origin of the fibrinogen or fibrin is blood plasma. More preferably, the blood plasma is of autologous origin. The composition of the step can likewise be prepared from a plasma derivative, such as, for example, but without being limited to, a cryoprecipitate or a fibrinogen concentrate. In addition to fibrinogen, the composition may contain other coagulation factors.

In a preferred embodiment, the concentration of fibrinogen in the resulting product is between 0.5 and 10 g/L, optionally between 1 and 10 g/L. In a more preferred embodiment, the concentration in the resulting product is between 1 and 4 g/L, optionally between 2 and 4 g/L. Nevertheless, a higher or lower concentration may also be used.

Antifibrinolytic Agent

The fibrin polymer can be degraded by means of the process referred to as fibrinolysis. During fibrinolysis, plasminogen is converted into the active enzyme, plasmin, by the tissue plasminogen activator; plasmin binds to the surface of fibrin through its binding sites to cause degradation of the fibrin polymer. To avoid fibrin matrix fibrinolysis, in step (b) of the present invention is added an antifibrinolytic agent such as, for example, but without being limited to, epsilon-aminocaproic acid, tranexamic acid, or aprotinin.

In another preferred embodiment of this aspect of the invention, the antifibrinolytic agent of (II) is tranexamic acid.

Tranexamic acid is a synthetic product derived from the amino acid lysine and has a high affinity for the plasminogen lysine-binding sites; it blocks these sites and prevents activated plasminogen from binding to the surface of fibrin, exerting an antifibrinolytic effect. Tranexamic acid has the advantage, compared to other antifibrinolytic agents of origin animal, of not transmitting diseases. Therefore, in a preferred embodiment, the antifibrinolytic agent is tranexamic acid. In an even more preferred embodiment, the concentration of tranexamic acid in the product resulting from step (e) is between 0.5 and 2 g/L, preferably between 1 and 2 g/L.

Nevertheless, a higher or lower concentration may also be used.

Source of Calcium

In another preferred embodiment of this aspect of the invention, the source of calcium is a calcium salt. More preferably, the calcium salt is calcium chloride.

The concentration of the calcium salt must be sufficient so as to induce fibrinogen polymerization. In a more preferred embodiment, the calcium salt is calcium chloride. In an even more preferred embodiment, the concentration of calcium chloride in the resulting product is between 0.25 and 3 g/L, optionally between 0.5 and 4 g/L. Nevertheless, a higher or lower concentration may also be used.

Polysaccharide

Fibrin matrices are quite versatile, so they have been used for producing different artificial tissues; however, the clinical use thereof has been limited fundamentally due to the fact that they present little consistency, are hard to handle, and are extremely fragile. For that reason, a polysaccharide is added. Said polysaccharide is generally used to provide resistance and consistency to the tissue, and it is convenient for it to be soluble therein. Examples of polysaccharides which can be used are, but without being limited to, agar-agar, agarose, alginate, chitosan, or carrageenans, or any combination thereof.

In another preferred embodiment of this aspect of the invention, the polysaccharide is agarose. Even more preferably, the agarose is type VII agarose.

Agarose is a polysaccharide formed by alpha and beta galactoses extracted from algae of genera such as Gelidium or Gracilaria. Compared to other polysaccharides which can be used in step (e) of the present invention, agarose presents the advantage that it forms an inert matrix from the immunological viewpoint. Therefore, in a preferred embodiment, the polysaccharide of the invention is agarose. There are different types of agarose the physical and chemical properties of which vary, such as, for example, gelling temperature, gel strength, and/or porosity. Preferably, the agarose is an agarose with a low melting point, i.e., an agarose that repolymerizes and solidifies at a temperature of preferably less than 65° C. and, more preferably, less than 40° C.; it can thereby be used to prepare the tissue at very low temperatures, minimizing the probability of cell death. In a more preferred embodiment, the agarose used is type VII agarose. In an even more preferred embodiment, the agarose, preferably type VII agarose, in the resulting product is advantageously at a concentration of between 0.1 and 6 g/L, optionally between 0.2 and 6 g/L, preferably between 0.15 and 3 g/L, optionally between 0.3 and 3 g/L, and more preferably between 0.25 and 2 g/L, optionally between 0.5 and 2 g/L.

Nevertheless, a higher or lower concentration may also be used.

Protein, Preferably Collagen

In another preferred embodiment, the biomaterial of the invention further comprises a protein. More preferably, the protein is selected from fibronectin, collagen, or the combination thereof. Even more preferably, the protein is collagen, and even more preferably type I collagen.

Examples of proteins which can be used are, but without being limited to, collagen, reticulin, or elastin. The addition of a protein gives rise to tissues having a higher fiber density at the stromal level, enhanced viscoelastic behavior, and an increasing threshold stress.

The main rheological properties of a solid or semisolid material are viscosity and elasticity. Viscosity is the resistance put up by a fluid against tangential strain, and would be equivalent to the consistency or rigidity. Elasticity is the mechanical property of certain materials of suffering reversible strain when subjected to the action of external forces, and of recovering their original shape when these external forces cease. The analysis of these parameters is performed by means of rheometry, a physical technique that uses instruments referred to as rheometers.

The stress threshold is the mechanical force required to cause irreversible strain in a solid or fluid. Normally all materials have an elastic region, in which the applied stress causes a strain that is completely reversible when the stress ceases. If that stress exceeds a limit (yield strength), the strain becomes irreversible, entering a plastic a plastic region. Finally, if the stress exceeds the plastic modulus, the material breaks (fracture point).

Collagen is a protein readily found in nature and biologically characterized by its low immunogenicity and high tissue activity. Collagen forms collagenous fibers, which are flexible, but offer considerable tensile strength. The artificial tissues of the present invention, which may contain fibrin, agarose, and collagen, have a higher fiber density at the stromal level, an enhanced viscoelastic behavior, and present an increasing threshold stress as the concentration of collagen increases, being higher than artificial collagen tissues. Therefore, in a preferred embodiment the added protein is collagen.

In a preferred embodiment, the added collagen is selected from the list comprising: type I collagen, type II collagen, type III collagen, type IV collagen, collagen type V, collagen type VI, collagen type VII, type VIII collagen, type IX collagen, type X collagen, type XI collagen, type XII collagen, type XIII collagen, or any combination thereof. In a more preferred embodiment, the added collagen is selected from the list comprising: type I collagen, type II collagen, type III collagen, type IV collagen, collagen type V, type IX collagen, or any combination thereof. The selection of a particular type of collagen depends on the artificial tissue to be prepared and is performed according to the characteristics of each collagen that are known in the state of the art.

For example, the main function of type I collagen is that of providing resistance to stretching, and is found abundantly in the dermis, bones, tendons, and corneas. The present invention thus demonstrates that the addition of type I collagen confers excellent properties to the artificial tissue for preparing, for example, but without limitation to, a substitute corneal tissue or an artificial cornea. Therefore, in a preferred embodiment the collagen is type I collagen. In an even more preferred embodiment, the collagen, preferably type I collagen, in the resulting product is advantageously at a concentration of between 0.5 and 5 g/L, preferably between 1.8 and 3.7 g/L, and more preferably between 2.5 and 3 g/L. Nevertheless, a higher or lower concentration may also be used.

In a particular embodiment, the collagen used is an atelocollagen, i.e., a collagen from which the terminal regions having a non-helical structure referred to as telopeptides have been removed. These telopeptides can render collagen insoluble and are carriers of the main antigenic determinants of collagen. Atelocollagen is obtained, for example, by means of protease treatment with pepsin.

Depending on the concentrations of fibrinogen that are used, the concentration of polysaccharide, and, in the event that it is used, the concentration of collagen that is used, the artificial tissue resulting may comprise variable concentrations of the two/three components.

In a preferred embodiment, in the resulting product the concentration of fibrinogen is between 0.5 and 10 g/L, the concentration of agarose, preferably type VII agarose, is between 0.1 and 6 g/L. If included, the concentration of collagen, preferably type I collagen, is between 0.5 and 5 g/L.

In another preferred embodiment, in the resulting product the concentration of fibrinogen is between 0.5 and 10 g/L, the concentration of agarose, preferably type VII agarose, is between 0.15 and 3 g/L. If included, the concentration of collagen, preferably type I collagen, is between 0.5 and 5 g/L.

In another preferred embodiment, in the resulting product the concentration of fibrinogen is between 0.5 and 10 g/L, the concentration of agarose, preferably type VII agarose, is between 0.25 and 2 g/L. If included, the concentration of collagen, preferably type I collagen, is between 0.5 and 5 g/L.

In another preferred embodiment, in the resulting product the concentration of fibrinogen is between 0.5 and 10 g/L, the concentration of agarose, preferably type VII agarose, is between 0.1 and 6 g/L. If included, the concentration of collagen, preferably type I collagen, is between 1.8 and 3.7 g/L.

In another preferred embodiment, in the resulting product the concentration of fibrinogen is between 0.5 and 10 g/L, the concentration of agarose, preferably type VII agarose, is between 0.15 and 3 g/L. If included, the concentration of collagen, preferably type I collagen, is between 1.8 and 3.7 g/L.

In another preferred embodiment, in the resulting product the concentration of fibrinogen is between 0.5 and 10 g/L, the concentration of agarose, preferably type VII agarose, is between 0.25 and 2 g/L. If included, the concentration of collagen, preferably type I collagen, is between 1.8 and 3.7 g/L.

In another preferred embodiment, in the resulting product the concentration of fibrinogen is between 0.5 and 10 g/L, the concentration of agarose, preferably type VII agarose, is between 0.1 and 6 g/L. If included, the concentration of collagen, preferably type I collagen, is between 2.5 and 3 g/L.

In another preferred embodiment, in the resulting product the concentration of fibrinogen is between 0.5 and 10 g/L, the concentration of agarose, preferably type VII agarose, is between 0.15 and 3 g/L. If included, the concentration of collagen, preferably type I collagen, is between 2.5 and 3 g/L.

In another preferred embodiment, in the resulting product the concentration of fibrinogen is between 0.5 and 10 g/L, the concentration of agarose, preferably type VII agarose, is between 0.25 and 2 g/L. If included, the concentration of collagen, preferably type I collagen, is between 2.5 and 3 g/L.

In another preferred embodiment, in the resulting product the concentration of fibrinogen is between 1 and 4 g/L, the concentration of agarose, preferably type VII agarose, is between 0.1 and 6 g/L. If included, the concentration of collagen, preferably type I collagen, is between 0.5 and 5 g/L.

In another preferred embodiment, in the resulting product the concentration of fibrinogen is between 1 and 4 g/L, the concentration of agarose, preferably type VII agarose, is between 0.15 and 3 g/L. If included, the concentration of collagen, preferably type I collagen, is between 0.5 and 5 g/L.

In another preferred embodiment, in the resulting product the concentration of fibrinogen is between 1 and 4 g/L, the concentration of agarose, preferably type VII agarose, is between 0.25 and 2 g/L. If included, the concentration of collagen, preferably type I collagen, is between 0.5 and 5 g/L.

In another preferred embodiment, in the resulting product the concentration of fibrinogen is between 1 and 4 g/L, the concentration of agarose, preferably type VII agarose, is between 0.1 and 6 g/L. If included, the concentration of collagen, preferably type I collagen, is between 1.8 and 3.7 g/L.

In another preferred embodiment, in the resulting product the concentration of fibrinogen is between 1 and 4 g/L, the concentration of agarose, preferably type VII agarose, is between 0.15 and 3 g/L. If collagen has been included, the concentration of the collagen, preferably type I collagen, is between 1.8 and 3.7 g/L.

In another preferred embodiment, the concentration of fibrinogen is between 1 and 4 g/L, the concentration of agarose, preferably type VII agarose, is between 0.25 and 2 g/L. If included, the concentration of collagen, preferably type I collagen, is between 1.8 and 3.7 g/L.

In another preferred embodiment, in the resulting product the concentration of fibrinogen is between 1 and 4 g/L, the concentration of agarose, preferably type VII agarose, is between 0.1 and 6 g/L. If included, the concentration of collagen, preferably type I collagen, is between 2.5 and 3 g/L.

In another preferred embodiment, in the resulting product the concentration of fibrinogen is between 1 and 4 g/L, the concentration of agarose, preferably type VII agarose, is between 0.15 and 3 g/L. If included, the concentration of collagen, preferably type I collagen, is between 2.5 and 3 g/L.

In another preferred embodiment, in the resulting product the concentration of fibrinogen is between 1 and 4 g/L, the concentration of agarose, preferably type VII agarose, is between 0.25 and 2 g/L. If included, the concentration of collagen, preferably type I collagen, is between 2.5 and 3 g/L.

As it is used herein, the term “coagulation factor” refers to a component, generally a protein, present in blood plasma and involved in the chain reaction enabling coagulation. There are thirteen coagulation factors named with Roman numerals: I: fibrinogen; II: prothrombin; III: tissue factor or thromboplastin; IV: calcium; V: proaccelerin; VI: inactive factor or zymogen; VII: proconvertin; VIII: antihemophilic factor A or von Willebrand factor; IX: antihemophilic factor B or Christmas factor; X: Stuart-Prower factor; XI: antihemophilic factor C; XII: Hageman factor; XIII: Fibrin stabilizing factor; XIV: Fitzgerald; XV: Fletcher; XVI: platelets; and XVII: Somocurcio. Preferably, the other coagulation factor added in step (c) of the method of the present invention is factor XIII.

In another preferred embodiment, the biomaterial of the invention further comprises another active ingredient. As it is used throughout the present specification, the term “active ingredient” has the same meaning as “active substance”, “pharmaceutically active substance”, “active component” or “pharmaceutically active component”, and refers to any component potentially providing a pharmacological activity or another different effect on the diagnosis, cure, mitigation, treatment, or prevention of a disease, or affecting the structure or function of the body of humans or other animals. The term includes those components promoting a chemical change in the production of the drug, and they are present in same in an expected modified form providing the specific activity or the effect.

A preferred embodiment of this aspect of the invention relates to the biomaterial of the invention for use as a medicinal product, or alternatively, to use of the biomaterial of the invention in the production of a medicinal product.

Another preferred embodiment of this aspect of the invention relates to the biomaterial of the invention for use in increasing, restoring, or partially or completely substituting the functional activity of a diseased or damaged tissue or organ, or alternatively to use of the biomaterial of the invention in the production of a medicinal product for increasing, restoring, or partially or completely substituting the functional activity of a diseased or damaged tissue or organ.

Artificial Tissue of the Invention

A second aspect of the invention relates to an artificial tissue, hereinafter artificial tissue of the invention, comprising the biomaterial of the invention, and further comprising mammalian cells.

In a more preferred embodiment of this aspect of the invention, the mammalian cells are human cells.

In a more preferred embodiment of this aspect of the invention, the artificial tissue of the invention consists of:
a) Fibrinogen (or fibrin) as previously described;
b) an antifibrinolytic agent, preferably as previously described;
c) an element selected from: a coagulation factor, a source of calcium as previously described;
d) a polysaccharide as previously described;
e) a compound of formula (I) as previously described; and
f) a mammalian cell, preferably a human cell.

Said cells can be obtained by means of different methods described in the state of the art, which may depend on the particular cell type involved. Some of these methods are, for example, but without being limited to, biopsy, mechanical processing, enzymatic treatment (for example, but without being limited to, with trypsin or type I collagenase), centrifugation, erythrocyte lysis, filtration, culture in supports or media favoring the selective proliferation of said cell type or immunocytometry.

The cells can be differentiated cells such as, for example, but without being limited to, fibroblasts, keratocytes, or smooth muscle cells, or undifferentiated cells with the capacity to differentiate into said cells such as, for example, adult stem cells.

Therefore, in another preferred embodiment of this aspect of the invention, the cells are selected from: keratinocytes, urothelial cells, epithelial cells of the urethra, corneal epithelial cells, epithelial cells of the oral mucosa, stromal cells, glial cells, neuronal cells, stem cells, or any combinations thereof.

In a preferred embodiment of the method of the invention, the cells are fibroblasts or undifferentiated cells with the capacity to differentiate into fibroblasts. The fibroblasts can be obtained from any tissue or organ; however, the fibroblasts preferably come from the tissue or of the organ in which the artificial tissue is to be used as a substitute. For example, when the method of the invention is used for preparing a substitute skin tissue or an artificial skin, the fibroblasts preferably come from the skin (dermal fibroblasts); when it is used for preparing a substitute bladder tissue or an artificial bladder, the fibroblasts preferably come from the bladder; when it is used for preparing a substitute urethra tissue or an artificial urethra the fibroblasts preferably come from the urethra; or when it is used for preparing a substitute oral mucosa tissue or an artificial oral mucosa, the fibroblasts preferably come from oral mucosa.

Nevertheless, the fibroblasts can be obtained from any other tissue or organ, such as, for example, the oral mucosa, the abdominal wall, or any connective tissue. For example, the fibroblasts obtained from oral mucosa can be used for preparing a substitute skin tissue or an artificial skin, a substitute bladder tissue or an artificial bladder, a substitute urethra tissue or an artificial urethra, or a substitute corneal tissue or an artificial cornea.

Therefore, in a preferred embodiment of this aspect of the invention, the fibroblasts come from the stroma of a tissue or an organ selected from the list comprising: oral mucosa, abdominal wall, skin, bladder, urethra, or cornea.

In another preferred embodiment of the method of the invention, the cells are keratocytes or undifferentiated cells with the capacity to differentiate into keratocytes. For example, when the method of the invention is used for preparing a substitute corneal tissue or an artificial cornea, preferably keratocytes of the corneal stroma are used.

The possibility of all the components of the artificial tissue being of autologous origin allows being able to transplant said tissue without requiring immunosuppression of the transplanted subject. However, the components of the artificial tissue can also be of allogeneic origin, i.e., they may come from an individual other than the one in whom the artificial tissue is to be transplanted. Even the species from which said components come may be different; in that case, their origin is said to be xenogeneic. This opens up the possibility of the artificial tissue being prepared beforehand when it is needed urgently, although in this case it would be advisable to apply immunosuppression of the subject in which the artificial tissue is transplanted.

Therefore, in a preferred embodiment the cells of step (a) of the invention are of autologous origin. Nevertheless, the cells of step (a) can also be of allogeneic or xenogeneic origin.

After the addition of the cells to the biomaterial of the invention, and after leaving the resulting product to stand in a support, the formation of a matrix comprising fibrin, the polysaccharide, and the compound of formula (I) is performed, in which matrix said cells are embedded and/or deposited and on and/or within which matrix such cells can grow. Preferably, the cells of step (a) grow within said matrix.

As demonstrated in the examples of the present invention, the addition of the compound of formula (I) allows preparing hydrogels with mechanical properties that can be regulated by up to an order of magnitude by suitably choosing the concentration of said compound of formula (I).

In another preferred embodiment of this aspect of the invention, the stem cells are selected from mesenchymal stem cells, hematopoietic stem cells, embryonic stem cells, induced pluripotent stem cells, adult stem cells, or combinations thereof.

The term “stem cell” refers to a cell with clonogenic capacity and the capacity to self-renew and differentiate into multiple cell lines. In particular, mesenchymal stem cells have the capacity to proliferate extensively and form fibroblastic cell colonies. As it is used in the present invention, the expression “stem cell” refers to a pluripotent or multipotent cell capable of generating one or more types of differentiated cells and furthermore exhibiting the capacity to self-renew, i.e., to make more stem cells. “Totipotent stem cells” can give rise to both embryonic components (such as, for example, the three embryonic layers, the germ line, and the tissues that will give rise to the yolk sac), and to extraembryonic components (such as the placenta). That is, they can form all cell types and give rise to a complete organism. “Pluripotent stem cells” can form any type of cell corresponding to the three embryonic lines (endoderm, ectoderm, and mesoderm), as well as the germ line and yolk sac. Therefore, they can form cell lines but a complete organism cannot be formed from them. “Multipotent stem cells” are those which can only generate cells of the same embryonic layer or line of origin. Bone marrow hosts at least two different stem cell populations: mesenchymal stem cells (MSCs) and hematopoietic stem cells (HSCs). In the context of the present invention, the stem cells are selected from the group comprising mesenchymal stem cells, hematopoietic stem cells, embryonic stem cells, induced pluripotent stem cells, adult stem cells, or combinations thereof. In a particular embodiment, the stem cells are mammalian stem cells, preferably human stem cells. In a particular embodiment, the stem cells are mesenchymal stem cells, preferably human mesenchymal stem cells.

The term “adult stem cell” refers to that stem cell which is isolated from a tissue or organ of an animal in a stage of growth after the embryonic stage. Preferably, the stem cells of the invention are isolated in a postnatal stage. They are preferably isolated from a mammal, and more preferably from a human, including newborns, children, adolescent, and adults. Adult stem cells can be isolated from a wide range of tissues and organs, such as bone marrow (mesenchymal stem cells, multipotent adult progenitor cells and hematopoietic stem cells), fatty tissue, cartilage, epidermis, hair follicle, skeletal muscle, heart muscle, intestine, liver, neurons.

The term “embryonic stem cell” or “ESC” are cells derived from an internal cell mass of embryos in the blastocyst stage, with the capacity to self-renew and differentiate into all types of adult cells. Embryonic stem cells are capable of proliferating indefinitely in vitro, being kept in an undifferentiated state and with a normal karyotype through prolonged culture. They also have the capacity to differentiate into cells of the three embryonic germ layers (mesoderm, endoderm, and ectoderm; (Itskovitz-Eldor, et al., Mol. Med. 6:88-95, 2000) and germ line. Embryonic stem cells represent a model that can be widely applied for research into the underlying mechanisms of pluripotent cell biology and differentiation into the early embryo, and they also provide opportunities for genetic manipulation. Embryonic stem cells have been isolated from the ICM of embryos in the blastocyst stage of a number of species (Bhattacharya, et al., BMC Dev. BioL 5:22, 2005), including mice (Solter and Knowles, Proc. Nati. Acad. USA 75:5565-5569, 1978.), pigs (Chen, et al., Theriogenology 52:195-212, 1999), and non-human primates (Thomson, et al., Proc. Nati. Acad. USA 92, 7844-7848, 1995).

The invention contemplates the use of embryonic stem cells coming from established cell lines of murine origin, such as lines 59B5, 36.5, 9TR #1, TK #1, ES-D3 [D3], YS001, ES-E14TG2a, ES-D3, 10P12, 56B3, L Wnt-3A, OP9, 3T3 MEFs WT, 3T3 MEFs KO, 127TAg, 151 TAg, WPE-stem, NE-4C, NE-GFP-4C, ES-C57BL/6, J1, R1, RW.4, B6/BLU, SCC #10, EDJ #22, AB2.2, Ainv15, 7AC5/EYFP, R1/E, G-Olig2, CE-1, CE3, and hESC BGO1 V, which are all available in public repositories.

Methods for obtaining embryonic stem cells are well known and can be put into practice by the expert without the need for excessive experimentation. Thus, human embryonic cells can be obtained as described in Reprod. Biomed. Online 4 (2002), 58-63. Embryonic cells of primates can be isolated from blastocysts of different primate species (Thomson et al., Proc. Nati. Acad. Sci. USA, 92:7844). Embryonic germ cells can be prepared from primordial germ cells present in human fetuses 8-11 weeks after the last menstrual period using methods such as that described by Shamblott et al., Proc. Nati. Acad. Sci. USA 95 (1998), 13726.

To avoid using human embryos, it is possible to use non-human transgenic animals as a source of embryonic stem cells. In particular, U.S. Pat. No. 5,523,226 describes methods for generating transgenic pigs that can be used as donors for xenotransplants in humans. WO97/12035 describes methods for producing transgenic animals suitable for xenotransplants. Likewise, WO01/88096 describes immunocompatible animal tissues. These immunocompatible animals can be used to generate pluripotent embryonic cells as described in U.S. Pat. No. 6,545,199.

Likewise, it is possible to use embryonic stem cell lines, which can be of a different origin. In one embodiment, the cell lines are from mice and include cells such as line R1 (ATCC No. SCRC-101 1) described by Nagy et al., (Proc. Natl. Acad. Sci. USA, 1993, 90:8424-8428) and the cell line D3.

For the case of countries where required by regulation, stem cells used in the invention have not been obtained by methods which destroy human embryos, or where appropriate, which use human embryos for industrial or commercial purposes. In another preferred embodiment of the invention, the stem cells are not embryonic stem cells.

The term “hematopoietic stem cell” or “HSC” refers to an adult stem cell with the capacity to give rise to both myeloid (monocytes and macrophages, neutrophils, basophils, eosinophils, erythrocytes, megakaryocytes/platelets, dendritic cells) and lymphoid (T lymphocytes, B cells, NK cells) hematopoietic cell lines. This cell type is fundamentally found in bone marrow.

As it is used herein, the term “mesenchymal stem cell” or “MSC” refers to a multipotent stromal cell originating from the mesodermal germ layer, which can differentiate into a range of cell types, including osteocytes (bone cells), chondrocytes (cartilage cells), and adipocytes (fat cells). Markers expressed by mesenchymal stem cells include CD105 (SH2), CD73 (SH3/4), CD44, CD90 (Thy-1), CD71, and Stro-1, as well as adhesion molecules CD106, CD166, and CD29. Between the markers negative for MSCs (unexpressed markers) are hematopoietic markers CD45, CD34, CD14, and costimulatory molecules CD80, CD86, and CD40, as well as adhesion molecule CD31. MSCs can be obtained from, without being limited to, bone marrow, fatty tissue (such as subcutaneous fatty tissue), liver, spleen, testicles, menstrual blood, amniotic fluid, pancreas, periosteum, synovial membrane, skeletal muscle, dermis, pericytes, trabecular bone, human umbilical cord, lung, dental pulp, and peripheral blood. The MSCs according to the invention can be obtained from any of the aforementioned tissues, such as from bone marrow, subcutaneous fatty tissue, or umbilical cord. MSCs can be isolated from bone marrow by means of methods known to one skilled in the art. In general, said methods consist of isolating mononuclear cells by means of density gradient centrifugation (Ficoll, Percoll) of bone marrow aspirates, and subsequently seeding the isolated cells in tissue culture plates in medium containing fetal bovine serum. These methods are based on the capacity of the MSCs to adhere to plastic, such that while non-adherent cells are removed from the culture, adhered MSCs can be expanded in culture plates. The MSCs can also be isolated from subcutaneous fatty tissue following a similar method, known to one skilled in the art. A method for isolating MSCs of bone marrow or subcutaneous fatty tissue has been previously described (De la Fuente et al., Exp. Cell Res. 2004, Vol. 297: 313:328). In a particular embodiment of the invention, the mesenchymal stem cells are obtained from umbilical cord, preferably human umbilical cord.

Therefore, in another preferred embodiment of the invention, the mesenchymal stem cells are obtained from bone marrow, fatty tissue, liver, spleen, testicles, menstrual blood, amniotic fluid, pancreas, periosteum, synovial membrane, skeletal muscle, dermis, pericytes, trabecular bone, umbilical cord, lung, dental pulp, and peripheral blood.

The umbilical cord constitutes an interesting source of adult stem cells because unlike adult stem cells obtained from other sources, (a) the method for obtaining them is neither invasive nor painful, and (b) their proliferative capacity and differentiation potential do not decrease as a result of the aging process. Among the different sources of umbilical cord stem cells, the so-called umbilical cord Wharton's jelly stem cells stand out because of: (a) their high proliferation capacity and a rapid expansion in culture, and (b) the low expression of major histocompatibility complex class I and absence of expression of the major histocompatibility complex class II, Io making them good candidates for allogeneic cell therapy.

Therefore, in another preferred embodiment, the cells of step (f) are umbilical cord Wharton's jelly stem cells. These cells express on their surface various markers characteristic of mesenchymal cells such as, for example, SH2, SH3, CD10, CD13, CD29, CD44, CD54, CD73, CD90, CD105, or CD166, and are negative for markers of the hematopoietic line, such as, for example, CD31, CD34, CD38, CD40, or CD45. Umbilical cord Wharton's jelly stem cells can differentiate, for example, into chondroblasts, osteoblasts, adipocytes, neural precursor cells, cardiomyocytes, skeletal muscle cells, endothelial cells, or hepatocytes.

Adult stem cells can be characterized by means of identifying surface and/or intracellular proteins, genes, and/or other markers indicative of their undifferentiated state, by means of different methods known in the state of the art such as, for example, but without being limited to, immunocytometry, analysis immunocytochemical, Northern blot analysis, RT-PCR, gene expression analysis in microarrays, proteomic studies, or differential display analysis.

The stem cells can be induced to differentiate in vitro to give rise to cells expressing at least, one or more characteristics typical of differentiated cells. Examples of differentiated cells into which stem cells can differentiate are, but without being limited to, fibroblasts, keratinocytes, urothelial cells, epithelial cells of the urethra, corneal epithelial cells, epithelial cells of the oral mucosa, chondroblasts, osteoblasts, adipocytes, or neuronal cells. In a preferred embodiment of the invention, the cell differentiated from the multipotent stem cell of the invention expresses one or more characteristics typical of a differentiated cell selected from the list comprising: fibroblasts, keratinocytes, urothelial cells, epithelial cells of the urethra, corneal epithelial cells, epithelial cells of the oral mucosa, chondroblasts, osteoblasts, adipocytes, or neuronal cells.

The differentiated cells can be characterized by means of identifying surface and/or intracellular proteins, genes, and/or other markers indicative of their differentiated state, by means of different methods known in the state of the art such as, for example, but without being limited to, immunocytometry, analysis immunocytochemical, Northern blot analysis, RT-PCR, gene expression analysis in microarrays, proteomic studies, or differential display analysis.

The cells are left to proliferate until they reach a suitable number, typically until they reach at least 70% confluence, advantageously at least 80% confluence, preferably at least 90% confluence, more preferably at least 95% confluence, and even more preferably at least 100% confluence. During the time kept in culture, the culture medium in which the cells are located may be partially or completely replaced with new medium so as to replace the consumed ingredients and eliminate potentially damaging metabolites and catabolites.

The term “pluripotent stem cell” and grammatical equivalents are used interchangeably in the context of the present invention to refer to undifferentiated or slightly differentiated cells, of any species, with the capacity to divide indefinitely without losing their properties and which are capable of forming any cell of the three embryonic lines (mesoderm, endoderm, ectoderm) and germ line, as well as the germ line when cultured under certain conditions. The invention contemplates the use of any type of pluripotent stem cell that is capable of generating progeny of any of the three germ layers including cells derived from embryonic tissue, fetal, adult tissue, and other origins. Pluripotent cells suitable for use in the present invention include embryonic stem cells, embryonic carcinoma cells, induced pluripotent cells (iPSs) and primordial germ cells. Likewise, the invention contemplates the use of pluripotent stem cells of any species including, without limitation, human cells, mouse cells, rat cells, bovine cells, sheep cells, hamster cells, pig cells, and the like.

As used in the present invention, the term “induced pluripotent stem cell” or “iPS” refers to cells which are substantially identical from a genetic viewpoint to a differentiated somatic cell from which they derive, but which with regard to differentiation and proliferative capacity show characteristics similar to embryonic pluripotent stem cells. Typically, the iPSs express on their surface one or several markers selected from the group formed by SSEA-3, SSEA-4, TRA-I-60, TRA-1-81, TRA-2-49/6E, and Nanog. Typically, iPSs express one or several genes selected from the group of Oct-3/4, Sox2, Nanog, GDF3, REXI, FGF4, ESGI, DPP A2, DPP A4, and hTERT. iPSs can be generated using methods described in the state of the art, such as the methods described by Takahashi and Yamanaka (Cell, 2006, 126:663-676), Yamanaka et al. (Nature, 2007, 448:313-7), Wernig et al. (Nature, 2007, 448:318-24), Maherali (Cell Stem Cell, 2007, 1:55-70); Maherali and Hochedlinger (Cell Stem Cell, 2008, 3:595-605), Park et al. (Cell, 2008, 134:1-10); Dimos et al. (Science, 2008, 321:1218-1221), Blelloch et al. (Cell Stem Cell, 2007, 1:245-247); Stadtfeld et al. (Science, 2008, 322:945-949), and Okita et al. (Science, 2008, 322: 949-953). These cells are reprogrammed in vitro from somatic cells terminally differentiated by means of retroviral transduction of transcription factors Oct3/4, Sox2, Klf4, and c-Myc. Typically, iPS cells are obtained from somatic cells by means of the expression in said cells of proteins Oct-3/4 and Sox2, proteins Oct-3/4, Sox2 and Klf4, proteins Oct-3/4, Sox2, Klf4, and c-Myc, and/or proteins Oct-4, Sox2, Nanog, and LIN28.

By means of the addition of the cells to the different components, and after leaving the resulting product to stand in a support, the formation of a matrix comprising fibrin, the particles of the invention, the polysaccharide, the compound of the invention, and, where it has been included, the added protein is performed, in which matrix said cells are embedded and on and/or within which such cells can grow. Preferably, the cells grow within said matrix.

Supports which can be used are, for example, but without being limited to, tissue culture plates, or porous cell culture inserts. Preferably, said supports will be under conditions of sterility.

Medical Uses of the Invention

An infectious, inflammatory, genetic, or degenerative disease, physical or chemical damage, or an interruption to the blood flow, can give rise to a loss of cells in a tissue or organ. This cell loss could entail an alteration to the normal functioning of said tissue or organ, and therefore lead to the development of diseases or physical sequelae that lower the quality of life of a person. Therefore, it is important to try to regenerate or restore the normal functioning of said tissues or organs. The damaged tissue or organ can be substituted with new tissue or a new organ that has been produced in a laboratory by means of tissue engineering techniques. The objective of tissue engineering is the construction of artificial biological tissues and the use thereof for medical purposes so as to restore, substitute, or increase the functional activities of diseased tissues and organs. The therapeutic usefulness of techniques of this type is virtually unlimited, having applications in all fields. The use of tissue engineering techniques allows reducing waiting lists for tissues and organs, with the subsequent drop in morbidity and mortality of the disease in the recipient. It logically also results in a drop in morbidity and mortality in the organ donors. Moreover, there are a number of advantages associated with the use of autologous cells or tissues in tissue engineering, with the following standing out: (a) a significant reduction in the number of donor to recipient infections by infectious agents; and (b) the absence of graft versus host immune rejection, so the patient need not take any immunosuppressant treatment, whereby avoiding the side effects and problems associated with immunodepression.

Therefore, a third aspect of the invention relates to the use of the biomaterial or artificial tissue of the invention for increasing, restoring, or partially or completely substituting the functional activity of a diseased or damaged tissue or organ.

A more preferred embodiment relates to the use of the artificial tissue of the invention for increasing, restoring, or partially or completely substituting the functional activity of diseased or damaged skin as a result of a dysfunction, injury, or disease selected from the list comprising: a wound, an ulcer, a burn, a benign or malignant neoplasia, an infection, a contusion, a trauma, a chemical burn, or a birth defect.

A preferred embodiment of this third aspect relates to the use of the artificial tissue of the invention for increasing, restoring, or partially or completely substituting the functional activity of a bladder. A more preferred embodiment relates to the use of the artificial tissue of the invention for increasing, restoring, or partially or completely substituting the functional activity of a diseased or damaged bladder as a result of a dysfunction, injury, or disease selected from the list comprising: a benign or malignant neoplasia, an infection, a trauma, a birth defect (such as, for example, but without being limited to, exstrophy of the bladder, cloacal exstrophy, or contracted bladder), a neurogenic bladder, urinary incontinence, bladder dysfunction, bladder infection, or bladder stones.

A preferred embodiment of this third aspect relates to the use of the artificial tissue of the invention for increasing, restoring, or partially or completely substituting the functional activity of a urethra. A more preferred embodiment relates to the use of the artificial tissue of the invention for increasing, restoring, or partially or completely substituting the functional activity of a diseased or damaged urethra as a result of a dysfunction, injury, or disease selected from the list comprising: a benign or malignant neoplasia, an infection, a trauma, a birth defect (such as, for example, but without being limited to, hypospadias or epispadias), or stenosis.

A preferred embodiment of this third aspect relates to the use of the artificial tissue of the invention for increasing, restoring, or partially or completely substituting the functional activity of a cornea. A more preferred embodiment relates to the use of the artificial tissue of the invention for increasing, restoring, or partially or completely substituting the functional activity of a diseased or damaged cornea as a result of a dysfunction, injury, or disease selected from the list comprising: a corneal ulcer, a keratoconus, a keratoglobus, a descemetocele, a trauma, a chemical burn, limbal stem cell deficiency, atrophic keratitis, corneal dystrophy, a primary or secondary keratopathy, an infection, leukoma, a bullous keratopathy, a corneal endothelial cell dysfunction, or a benign or malignant neoplasia.

A preferred embodiment of this third aspect relates to the use of the artificial tissue of the invention for producing a medicinal product for increasing, restoring, or partially or completely substituting the functional activity of a mucosa, preferably an oral mucosa. An even more preferred embodiment relates to the use of the artificial tissue of the invention for increasing, restoring, or partially or completely substituting the functional activity of a diseased or damaged oral mucosa as a result of a dysfunction, injury, or disease selected from the list comprising: a wound, an ulcer, a burn, a benign or malignant neoplasia, an infection, a contusion, a trauma, a chemical burn, a birth defect, a substance loss, or a periodontal disease. In a preferred embodiment, the tissue used for increasing, restoring, or partially or completely substituting the functional activity of a mucosa is a tissue that has been subjected to a protein addition step. In an even more preferred embodiment, said step is carried out by means of the addition of a composition comprising collagen as indicated in detail above.

A fourth aspect of the invention relates to the use of the tissue of the invention in the production of a medicinal product, or alternatively to the biomaterial or the tissue of the invention for use in medicine.

Said medicinal product is a medicinal product for somatic cell therapy. “Somatic cell therapy” is understood to be the use of live, autologous, allogeneic, or xenogeneic somatic cells, the biological characteristics of which have been substantially altered as a result of their manipulation, for obtaining a therapeutic, diagnostic, or preventive effect, by metabolic, pharmacological, or immunological means. Among the medicinal products for somatic cell therapy are, for example, but without being limited to: cells manipulated for modifying their immunological, metabolic, or functional properties of another type in aspects qualitative or quantitative; cells which are sorted, selected, and manipulated, and subsequently subjected to a manufacturing process for the purpose of obtaining the end product; cells which are manipulated and combined with non-cellular components (for example, biological or inert matrices or medical devices) exerting the action that is sought in principle on the finished product; autologous cell derivatives expressed ex vivo (in vitro) under specific culture conditions; or cells which are genetically modified or subjected to another type of manipulation for expressing previously unexpressed homologous or non-homologous functional properties.

A fifth aspect of the invention relates to the use of the artificial tissue of the invention for producing a medicinal product for increasing, restoring, or partially or completely substituting the functional activity of a tissue or an organ. In a preferred embodiment, the damaged tissue or organ are selected from the list comprising: skin, bladder, urethra, cornea, mucosa, conjunctiva, abdominal wall, eardrum, pharynx, larynx, intestine, peritoneum, ligament, tendon, bone, meninx, or vagina.

A preferred embodiment of this aspect relates to the use of the artificial tissue of the invention for producing a medicinal product for increasing, restoring, or partially or completely substituting the functional activity of a diseased or damaged tissue or organ as a result of an infectious, inflammatory, genetic, or degenerative disease, physical or chemical damage, or an interruption to the blood flow.

A more preferred embodiment of this aspect relates to the use of the artificial tissue of the invention for producing a medicinal product for increasing, restoring, or partially or completely substituting the functional activity of skin. An even more preferred embodiment relates to the use of the artificial tissue of the invention for producing a medicinal product for increasing, restoring, or partially or completely substituting the functional activity of diseased or damaged skin as a result of a dysfunction, injury, or disease selected from the list comprising: a wound, an ulcer, a burn, a benign or malignant neoplasia, an infection, a contusion, a trauma, a chemical burn, or a birth defect.

A more preferred embodiment of this aspect relates to the use of the artificial tissue of the invention for producing a medicinal product for increasing, restoring, or partially or completely substituting the functional activity of a bladder. An even more preferred embodiment of this aspect relates to the use of the artificial tissue of the invention for producing a medicinal product for increasing, restoring, or partially or completely substituting the functional activity of a diseased or damaged bladder as a result of a dysfunction, injury, or disease selected from the list comprising: a benign or malignant neoplasia, an infection, a trauma, a birth defect (such as, for example, but without being limited to, exstrophy of the bladder, cloacal exstrophy, or contracted bladder), a neurogenic bladder, urinary incontinence, bladder dysfunction, bladder infection, or bladder stones.

A more preferred embodiment of this aspect relates to the use of the artificial tissue of the invention for producing a medicinal product for increasing, restoring, or partially or completely substituting the functional activity of a urethra. An even more preferred embodiment of this aspect relates to the use of the artificial tissue of the invention for producing a medicinal product for increasing, restoring, or partially or completely substituting the functional activity of a diseased or damaged urethra as a result of a dysfunction, injury, or disease selected from the list comprising: a benign or malignant neoplasia, an infection, a trauma, a birth defect (such as, for example, but without being limited to, hypospadias or epispadias), or stenosis.

A more preferred embodiment of this aspect relates to the use of the artificial tissue of the invention for producing a medicinal product for increasing, restoring, or partially or completely substituting the functional activity of a cornea. An even more preferred embodiment of this aspect relates to the use of the artificial tissue of the invention for producing a medicinal product for increasing, restoring, or partially or completely substituting the functional activity of a diseased or damaged cornea as a result of a dysfunction, injury, or disease selected from the list comprising: a corneal ulcer, a keratoconus, a keratoglobus, a descemetocele, a trauma, a chemical burn, limbal stem cell deficiency, atrophic keratitis, corneal dystrophy, a primary or secondary keratopathy, an infection, leukoma, a bullous keratopathy, a corneal endothelial cell dysfunction, or a benign or malignant neoplasia.

A more preferred embodiment of this aspect relates to the use of the artificial tissue of the invention for producing a medicinal product for increasing, restoring, or partially or completely substituting the functional activity of a mucosa, preferably an oral mucosa. An even more preferred embodiment relates to the use of the artificial tissue of the invention for producing a medicinal product for increasing, restoring, or partially or completely substituting the functional activity of a diseased or damaged oral mucosa as a result of a dysfunction, injury, or disease selected from the list comprising: a wound, an ulcer, a burn, a benign or malignant neoplasia, an infection, a contusion, a trauma, a chemical burn, a birth defect, a substance loss, or a periodontal disease. In a preferred embodiment, the tissue for producing a medicinal product for increasing, restoring, or partially or completely substituting the functional activity of a mucosa is a tissue that has been subjected to the addition of a protein. In an even more preferred embodiment, said step is carried out by means of the addition of a composition comprising collagen to the material as indicated in detail above.

A sixth aspect of the invention relates to a pharmaceutical composition comprising the artificial tissue of the invention.

A preferred embodiment of this aspect of the invention relates to a pharmaceutical composition comprising the artificial tissue of the invention for use in somatic cell therapy.

A more preferred embodiment of this aspect of the invention relates to a pharmaceutical composition comprising the artificial tissue of the invention for increasing, restoring, or partially or completely substituting the functional activity of a tissue or an organ. Preferably, the damaged tissue or organ is selected from: cornea, skin, oral mucosa, peripheral nerve, bladder, and cartilage.

A preferred embodiment of this aspect of the invention relates to a pharmaceutical composition comprising the artificial tissue of the invention for increasing, restoring, or partially or completely substituting the functional activity of a diseased or damaged tissue or organ as a result of an infectious, inflammatory, genetic, or degenerative disease, physical or chemical damage or an interruption to the blood flow.

A more preferred embodiment of this aspect of the invention relates to a pharmaceutical composition comprising the artificial tissue of the invention for increasing, restoring, or partially or completely substituting the functional activity of skin. An even more preferred embodiment relates to a pharmaceutical composition comprising the artificial tissue of the invention for increasing, restoring, or partially or completely substituting the functional activity of diseased or damaged skin as a result of a dysfunction, injury, or disease selected from the list comprising: a wound, an ulcer, a burn, a benign or malignant neoplasia, an infection, a contusion, a trauma, a chemical burn, or a birth defect.

A more preferred embodiment of this aspect of the invention relates to a pharmaceutical composition comprising the artificial tissue of the invention for increasing, restoring, or partially or completely substituting the functional activity of a bladder. An even more preferred embodiment relates to a pharmaceutical composition comprising the artificial tissue of the invention for increasing, restoring, or partially or completely substituting the functional activity of a diseased or damaged bladder as a result of a dysfunction, injury, or disease selected from the list comprising: a benign or malignant neoplasia, an infection, a trauma, a birth defect (such as, for example, but without being limited to, exstrophy of the bladder, cloacal exstrophy, or contracted bladder), a neurogenic bladder, urinary incontinence, bladder dysfunction, bladder infection, or bladder stones.

A more preferred embodiment of this aspect of the invention relates to a pharmaceutical composition comprising the artificial tissue of the invention for increasing, restoring, or partially or completely substituting the functional activity of a urethra. An even more preferred embodiment relates to a pharmaceutical composition comprising the artificial tissue of the invention for increasing, restoring, or partially or completely substituting the functional activity of a diseased or damaged urethra as a result of a dysfunction, injury, or disease selected from the list comprising: a benign or malignant neoplasia, an infection, a trauma, a birth defect (such as, for example, but without being limited to, hypospadias or epispadias), or stenosis.

A more preferred embodiment of this aspect of the invention relates to a pharmaceutical composition comprising the artificial tissue of the invention for increasing, restoring, or partially or completely substituting the functional activity of a cornea. An even more preferred embodiment relates to a pharmaceutical composition comprising the artificial tissue of the invention for increasing, restoring, or partially or completely substituting the functional activity of a diseased or damaged cornea as a result of a dysfunction, injury, or disease selected from the list comprising: a corneal ulcer, a keratoconus, a keratoglobus, a descemetocele, a trauma, a chemical burn, limbal stem cell deficiency, atrophic keratitis, corneal dystrophy, a primary or secondary keratopathy, an infection, leukoma, a bullous keratopathy, a corneal endothelial cell dysfunction, or a benign or malignant neoplasia.

A more preferred embodiment of this aspect relates to a pharmaceutical composition comprising the artificial tissue of the invention for producing a medicinal product for increasing, restoring, or partially or completely substituting the functional activity of a mucosa, preferably an oral mucosa. An even more preferred embodiment relates to a pharmaceutical composition comprising the artificial tissue of the invention for increasing, restoring, or partially or completely substituting the functional activity of a diseased or damaged oral mucosa as a result of a dysfunction, injury, or disease selected from the list comprising: a wound, an ulcer, a burn, a benign or malignant neoplasia, an infection, a contusion, a trauma, a chemical burn, a birth defect, a substance loss, or a periodontal disease. In a preferred embodiment, the pharmaceutical composition comprising the artificial tissue of the invention for producing a medicinal product for increasing, restoring, or partially or completely substituting the functional activity of a mucosa or an oral mucosa is a tissue to which a protein has been added. In an even more preferred embodiment, said step is carried out by means of the addition of a composition comprising collagen to the material obtained as indicated in detail above.

In a preferred embodiment of this aspect of the invention, the pharmaceutical composition comprises the artificial tissue of the invention, and furthermore, a pharmaceutically acceptable vehicle. In another preferred embodiment of this aspect of the invention, the pharmaceutical composition comprises the artificial tissue of the invention, and furthermore, another active ingredient. In a preferred embodiment of this aspect of the invention, the pharmaceutical composition comprises the artificial tissue of the invention and, furthermore, together with a pharmaceutically acceptable vehicle, another active ingredient.

The pharmaceutical compositions of the present invention can be used in a method of treatment alone or together with other pharmaceutical compounds.

Method for Obtaining the Biomaterial and the Tissue of the Invention

A seventh aspect of the invention relates to a method for producing the artificial tissue of the invention comprising:
a) adding an antifibrinolytic agent to a composition comprising fibrinogen (or fibrin);
b) adding at least a coagulation factor, a source of calcium, thrombin, or any combination thereof to the product resulting from step (a);
c) adding a composition comprising a polysaccharide to the product resulting from step (b), and leaving to gel;
d) subjecting the product resulting from step (c) to a controlled nanostructuring process;
e) inducing the cross-linking of the resultant of step (b) and/or (d) with a compound of formula (I) as described in the first aspect of the invention;
f) seeding the products of step (c) and/or (e) with mammalian cells.

For the correct differentiation of some cell types, an additional step may be needed. For example, in the case of epithelial cells of the oral mucosa, keratinocytes, or corneal epithelial cells, it may be necessary to expose the epithelial surface to air to promote correct stratification and maturation of the epithelium by keeping the matrix comprising the cells of step (a) immersed in culture medium (air-liquid technique).

Therefore, in a preferred embodiment, in addition to the steps (a)-(g) described above, the method of the invention comprises an additional step in which the product resulting from step (f) is exposed al air. In general, the method of the invention includes this step when used for obtaining an artificial tissue serving to replace a natural tissue the epithelium of which is usually exposed to contact with the air such as, for example, but without being limited to, the skin, the cornea, the oral mucosa, the urethra, or the vagina. Preferably, this step is performed when a substitute skin tissue or artificial skin is prepared, or when a substitute corneal tissue or an artificial cornea is prepared, or when a substitute oral mucosa tissue or an artificial oral mucosa is prepared.

Throughout the description and claims, the term “comprises” and variants thereof do not intend to exclude other technical features, additives, components, or steps. For those skilled in the art, other objects, advantages, and features of the invention will be inferred in part from the description and in part from putting the invention into practice. The following figures and examples are provided by way of illustration and are not intended to limit the present invention.

### EXAMPLES OF THE INVENTION

The following specific examples provided in this patent document serve to illustrate the nature of the present invention. These examples are included solely for illustrative purposes and must not be interpreted as limitations to the invention that is herein claimed. Therefore, the examples described below illustrate the invention without limiting the field of application thereof.

In Vitro Method for Preparing Artificial Tissue

The artificial tissue of the invention was prepared as described in the following steps:
a) adding a composition comprising fibrinogen human (purified plasma);
b) adding an antifibrinolytic agent (tranexamic acid) to the product resulting from step (a);
c) adding at least a coagulation factor, a source of calcium, thrombin, or any combination thereof to the product resulting from step (b);
d) adding a composition comprising a polysaccharide (agarose or similar) to the product resulting from step (c), and leaving to gel;
e) subjecting the product resulting from step (c) to a controlled nanostructuring process;
f) inducing the cross-linking of the resultant of step (c) and/or (e) with Genipin;
g) seeding the products of step (c) and/or (e) with human cells or cells of an experimental animal model.

The resulting tissue was subjected to a series of characterizations and tests described below.

Results

Biomechanical Characterization (Rheology):

The mechanical properties of the tissue samples were measured 24 hours after their preparation at 37° C. with a Haake MARS III controlled stress rheometer (Thermo Fisher Scientific, USA). The measurement geometry used was parallel plates, which consisted of two discs with a diameter of 3.5 cm, where the surface in contact with the sample presented certain roughness to prevent sliding phenomena on the surface. The tissue samples were gelled in 6-well culture plates with a diameter of 3.5 cm, the same as that of the plates of the rheometer. The method used was the following: the tissue sample is placed on the lower plate of the rheometer, and the upper plate is gradually moved closer until the normal force is slightly greater than 0 N (about 0.05 N). The distance between the two plates of the measurement system for which said value of normal force was attained varied from one sample to another, where it was between 2.5-5.8 mm for non-nanostructured gels (FAH) and between 50-400 μm for nanostructured gels (NFAH).

The biomechanical consistency of the hydrogels generated was evaluated both in steady state and in dynamic state, using the following protocols:

(i) Steady state. In this type of test, the sample is subjected to a shear stress ramp, the shear force needed to keep the strain in steady state being measured for each value. In the experiments that were performed, the application of each value of strain was maintained for 10 s. This process was repeated for increasing values of strain (strain ramp) until reaching the area of non-linear stress-strain dependence. A typical stress dependence with the shear strain for experiments of this type is shown in FIG. 1. As can be observed, at low values of strain, there is an approximately linear stress dependence with strain. As the applied strain increases, linearity starts to be lost, the curve taking on a concave shape, indicating that the successive strain of the material is easier as said material is more strained, i.e., the stress increments for an identical strain increment are lower as strain increases. This is the typical behavior of real elastic materials. The consistency of a material in steady state is usually quantified with the value of its modulus of rigidity, which is the slope of the linear area in the shear force vs. shearing strain curves.

(ii) Dynamic state. In tests of this type, the sample is subjected to an oscillatory shear force with a given frequency and amplitude and the resulting shear force is measured. Sinusoidal oscillatory stresses are usually applied, and, in the so-called linear viscoelastic region, the resulting stress is also sinusoidal, with the same frequency as the strain, but out-of-phase with respect to same [Macosko, 1994]. Under these conditions, the stress can be broken down into the in the sum of the part in phase with the strain and the part in phase opposition with same. Viscoelastic moduli G′ (elastic modulus) and G″ (viscous modulus) can thereby de defined as follows: G′ being the ratio between the amplitude of the part of the stress in phase with the strain and the amplitude thereof, and G″ being the ratio between the amplitude of the part of the stress in phase opposition with the strain and the amplitude thereof. Within the linear viscoelastic region (LVR), G′ quantifies the elastic response of the material, whereas G″ quantifies its viscous response. Two types of tests, that is, strain amplitude sweeps at constant frequency and frequency sweeps at constant amplitude, are carried out for dynamic state characterization.

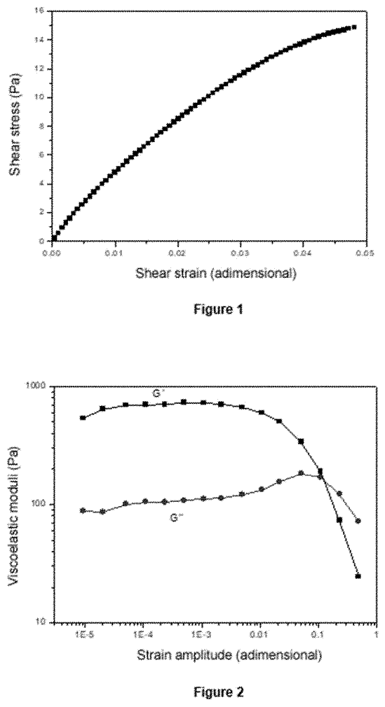

a. Amplitude sweep at constant frequency. During this test, the sample was subjected to a shearing strain of increasing amplitude, with the frequency being kept constant and equal to 1 Hz. For each value of amplitude, 5 oscillation cycles were performed, taking the mean of the last three cycles, discarding the first 2 so as to eliminate transients. A typical dependence of G′ and G″ in these experiments is shown in FIG. 2. As can be observed, both G′ and G″ have an initial value (low values of strain amplitude) approximately independent of the strain amplitude, with G′ rapidly decreasing for values of strain amplitude greater than about 0.01. This sudden decrease of G′ marks the start of the loss of linearity of the material. It is common to characterize the viscoelastic response of a material from the values of G′ and G″ corresponding to the LVR (flat region). It must be taken into account that the distinctly elastic materials (for example skin, elastomers, and rubber) have values of G′ that are much greater than those of G″ in the LVR, whereas the distinctly liquid materials (oils, fats, aqueous solutions, blood, etc.) have values of G″ greater than those of G′ in the LVR. The samples of the present invention are all distinctly elastic materials, with values of G′ that are much greater than those of G″.

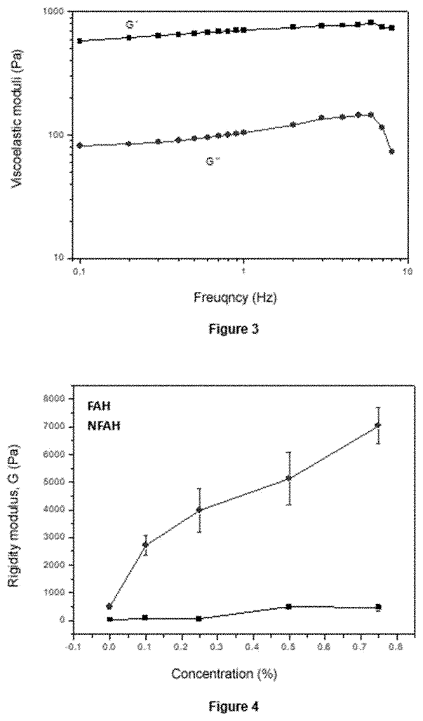

b. Frequency sweep at constant amplitude. In these tests in dynamic state, the material is subjected to oscillatory stresses with constant of amplitude (belonging to the LVR) and variable frequency. For each value of amplitude, 5 oscillation cycles were performed, taking the mean of the last three cycles, discarding the first 2 so as to eliminate transients. A typical dependence of the trends obtained is shown in FIG. 3. As can be observed in said, Figure, G′ and G″ have a slightly upward trend as the frequency of oscillation increases, with G′ being much greater than G″ throughout the entire range of values. This trend is typical of polymeric materials with high cross-linking [Macosko, 1994]. It must further be noted that G″ has a distinct downward trend at high values of frequency, which seems to be typical of human biological tissues [Rodriguez et al. Cryobiology 67 (2013) 355-362].

All the test mentioned were carried out with different aliquot of each type of sample. The results described below always correspond to the mean values obtained with at least 3 different aliquots.

The effect of the chemical agent Genipin on the mechanical properties of the prepared hydrogels will be analyzed below. Only the values for modulus of rigidity G, and viscoelastic moduli G′ and G″ corresponding to the LVR are shown herein (FIGS. 4, 5 and 6; Tables 1 and 2).

TABLE 1
Value of the modulus of rigidity, elastic modulus and viscous
modulus corresponding to the linear viscoelastic region
(corresponding to a shearing strain of 0.01 and a frequency
of 1 Hz) of the non-nanostructured hydrogels. Concentration
is indicated in % v/v of chemical agent Genipin.

| Non- | Elastic | Viscous | Modulus of |
|---|---|---|---|
| nanostructured | modulus | modulus | rigidity |
| gels | G′ (Pa) | G″ (Pa) | G (Pa) |
| FA-Ctr | 36 ± 7 | 8.9 ± 1.7 | 31.5 ± 2.3 |
| Gen 0.1 | 93 ± 8 | 25 ± 3 | 84 ± 8 |
| Gen 0.25 | 74 ± 8 | 10.3 ± 1.0 | 65 ± 4 |
| Gen 0.5 | 610 ± 40 | 124 ± 8 | 490 ± 9 |
| Gen 0.75 | 570 ± 160 | 121 ± 19 | 470 ± 120 |

TABLE 2
Value of the modulus of rigidity, elastic modulus and viscous
modulus corresponding to the linear viscoelastic region
(corresponding to a shearing strain of 0.01 and a frequency
of 1 Hz) of the nanostructured hydrogels. Is indicated the
concentration in % v/v of chemical agent Genipin.

| | Elastic | Viscous | Modulus of |
|---|---|---|---|
| Nanostructured | modulus | modulus | rigidity |
| gels | G′ (Pa) | G″ (Pa) | G (Pa) |
| FA-Ctr | 1130 ± 220 | 350 ± 90 | 500 ± 60 |
| Gen 0.1 | 3400 ± 400 | 560 ± 40 | 2700 ± 300 |
| Gen 0.25 | 3900 ± 400 | 900 ± 140 | 4000 ± 800 |
| Gen 0.5 | 7400 ± 1000 | 2600 ± 600 | 5100 ± 1000 |
| Gen 0.75 | 10600 ± 1700 | 2200 ± 600 | 7000 ± 600 |

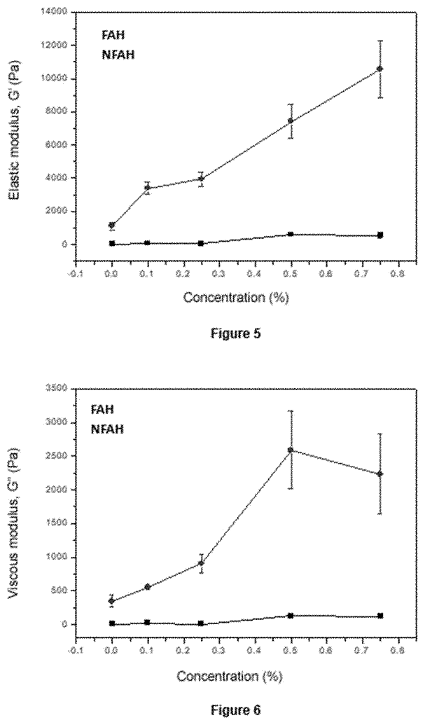

As can be observed by comparing FIGS. 5 and 6 and columns 1 and 2 of Tables 1 and 2, in all cases the values of G′ are much greater than the values of G″, so all the hydrogels have distinctly elastic properties, characteristic of a cross-linked polymer material. Furthermore, for all the samples the values of G′ are about 4 times greater than those of G″, and the amount of Genipin does not noticeably affect this ratio. Therefore, under external stresses, most of the energy communicated to the hydrogel would be stored in the form of elastic energy (corresponding to elastic modulus G′), whereas a small part would be dissipated by viscous drag (corresponding to the viscous modulus). With regard to the effect of Genipin, it is observed to consist of a gradual increase of both the modulus of rigidity and the viscoelastic moduli, reaching values up to one order of magnitude higher (for G and G′ as well as G″) for the maximum concentration of Genipin with respect to the control hydrogel (absence of Genipin), both for the non-nanostructured hydrogels and for the nanostructured hydrogels. As can be observed, the regulation of the mechanical properties is progressive when going from the control hydrogel to the hydrogel containing Genipin 0.75, so it is possible to prepare hydrogels with mechanical properties that can be regulated by up to an order of magnitude by suitably choosing the concentration of the chemical agent. Nevertheless, it must be observed that there is certain saturation of the mechanical properties for concentrations of Genipin greater than 0.5, which is especially evident in the case of non-nanostructured hydrogels.

With regard to the effect of nanostructuring, as can be observed by direct comparison of the data in FIGS. 4, 5 and 6, the nanostructured hydrogels have values of biomechanical parameters (G, G′ and G″) 20-30 times greater than those corresponding to the non-nanostructured hydrogels. It must furthermore be observed that the higher values of the mechanical parameters of the non-nanostructured hydrogels (achieved for the highest concentrations of Genipin) are about half the values of the control nanostructured hydrogels. In conclusion, through the effect of nanostructuring and of the concentration of Genipin, it is possible to regulate the properties of fibrin and agarose hydrogels in a range going from the values of the control non-nanostructured hydrogels up to 300 times the value of these hydrogels. Lastly, it must be noted that the range of values of the mechanical properties covered by the hydrogels of this invention encompass a wide range of natural human tissues, as can be seen by comparing with FIG. 1 of the paper [Scionti G, Moral M, Toledano M, Osorio R, Duran J D G, Alaminos M, Campos A, Lopez-Lopez M T. Effect of the hydration on the biomechanical properties in a fibrin-agarose tissue-like model. J Biomed Mater Res Part A 2014:102A:2573-2582]. As can be seen in said figure, a large number of native human tissues have values of G′ in the range of 1-10000 Pa, values of G″ in the range of 0.1-7000 Pa, and values of G in the range up to 10000 Pa. Said ranges are covered by the hydrogels of the present invention.

Structural Characterization:

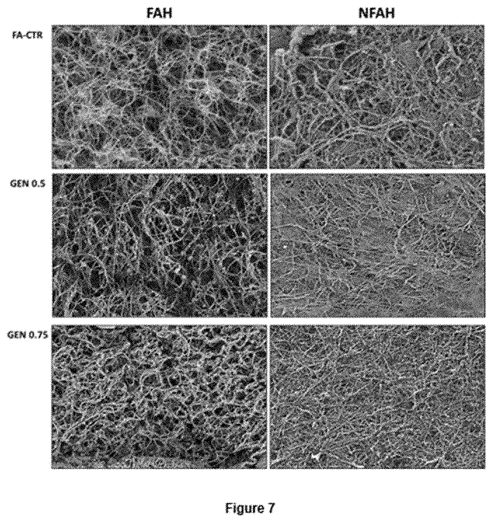

The structural studies carried out by scanning electron microscopy (FIG. 7) show the fiber composition characteristic of the non-nanostructured FA hydrogel (FAH) and nanostructured FA hydrogel (NFAH). The non-nanostructured gels are characterized by being made up of randomly organized fibers, whereas those gels subjected to a nanostructuring process showed a more organized pattern. In both cases, the high porosity of this biomaterial is conserved (FIG. 7).

The hydrogels subjected to cross-linking with Genipin showed a fiber and fiber organizational pattern comparable to the hydrogels without Genipin. However, this analysis demonstrates that the chemical reaction with Genipin induced a decrease in porosity of the biomaterials depending on the concentration of the chemical agent, as shown in the representative images of FIG. 7.

Ex Vivo Biocompatibility:

To determine the biocompatibility of the membranes generated in this invention, human fibroblasts were used, seeded on the surface of FAH and of those subjected to cross-linking with Genipin. A histological analysis and the live-dead cell viability assay (Gibco) were subsequently performed.

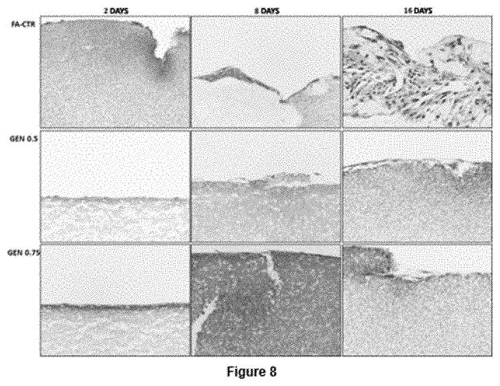

The histological analysis shows that the fibroblasts adhere to the surface of all the biomaterials. In the case of the FAH without cross-linking, a large amount of cells can be seen over the course of time (0-16 days ex vivo) which increase in number, enter the hydrogels and start a progressive degradation of their fibers. When the hydrogels subjected to cross-linking with Genipin were analyzed, it was observed that the cells adhere to the surface of the biomaterials. However, the fibroblasts showed a considerably lower degradation and invasion activity than the hydrogels without cross-linking (FIG. 8).

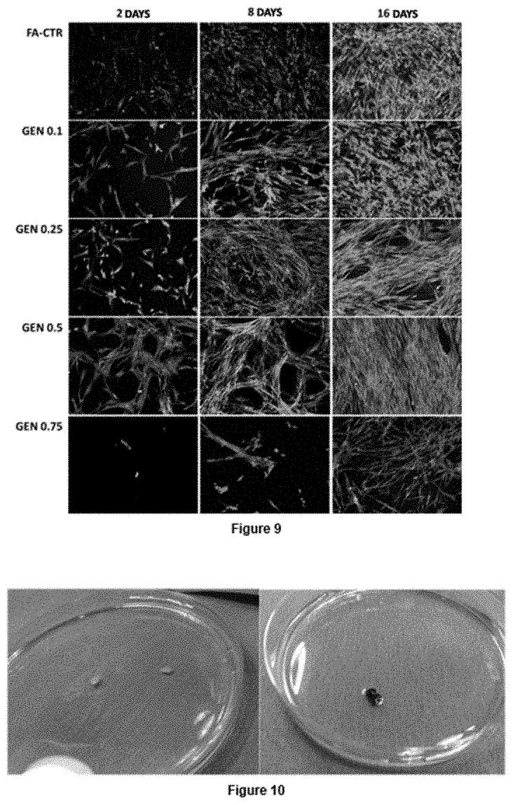

The (live-dead) cell viability assay showed a large amount of viable and metabolically active fibroblasts on the surface of all the biomaterials analyzed (FIG. 9). In the case of the FAH hydrogels without cross-linking (CTR), a large number of cells with a spindle-shaped morphology are observed after 2 days, which considerably increased in number after 16 days of ex vivo development. When the hydrogels subjected to cross-linking with Genipin were analyzed, it was observed that the fibroblasts adhere to the surface of the biomaterial and adopt their characteristic spindle-shaped morphology, especially at low concentrations of this agent. However, and as this test demonstrates, the cells cultured on the surface present difficulties in the cell adhesion process the first 2 days, but over the course of time they considerably increase in number in those hydrogels subjected to cross-linking with Genipin at 0.75% (FIG. 9). The dead cells are observed with red fluorescence. However, since dead cells lose their adhesion function, they are eliminated from the surface of the biomaterial when the culture medium is changed, so it is not possible to see them on the surface of these hydrogels.

Finally, the histological analyses and viability assay demonstrate that the FAH hydrogels subjected to cross-linking with different concentrations of Genipin are highly biocompatible and more resistant to the degradation on the part of the cells cultured on the surface. From this it can be concluded that the process of cross-linking FAH hydrogels with Genipin increases the biomechanical properties, maintains the biocompatibility properties, and delays the degradation mediated by the cells of these biomaterials. Therefore, the FAH membranes chemically modified with Genipin developed in this invention could be used safely in various applications in the field of TE.

Production of Fibrin-Agarose Hydrogels

FA constructs were generated in this study following previously described protocols. To prepare 30 ml of FA, 22.8 ml of plasma human (provided by Dr. Fernandez-Montoya, of the Centro Regional de Transfusion Sanguinea y Banco de Tejidos de Granada y Almeria, Regional Blood Transfusion and Tissue Bank Center of Granada and Almeria), 2.25 ml of PBS (with 5% antibiotic solution), and 450 μl of tranexamic acid (Amchafibrin®) were used. This solution was carefully mixed, and then 1.5 ml of type VII agarose and 3 ml of 2% calcium chloride (to promote gelling) were used, the solution was carefully mixed and distributed on 6-well plates (with a volume of 5 ml in each one). The FA constructs were left to gel for about 2 hours at 37° C. As a result, acellular FA constructs were obtained with a volume of 5 mL, 0.5 cm thick and 3.5 in diameter.

Once gelling of the FA constructs was completed, nanostructuring thereof was performed by means of plastic compression. To that end, the samples were placed between a pair of nylon filters (with a pore size of 0.22 μm) and subsequently compressed between a pair of sterile absorbent filters 3 mm thick. A weight of 500 g was subsequently applied for a period of 3 minutes, obtaining FA constructs 30 mm in diameter and between 50-400 μm thick.

Cross-Linking of Fibrin-Agarose Constructs

In this embodiment, FA constructs (FAH and NFAH) were subjected to cross-linking with 4 concentrations of Genipin (0.1%, 0.25%, 0.5%, 0.75%). In this sense, 5 ml of Genipin were added to each well, covered with aluminum foil, and incubated for 72 hours at 37° C. After this time, the cross-linking reaction was verified by the blue coloring of the construct, and washing with PBS with 5% antibiotic was subsequently performed during 24 hours.

Biological Membranes

Biological membranes were generated from fibrin-agarose hydrogels (FAHs) with controlled biomechanical properties. The FAHs were subjected to a chemical cross-linking process by immersion in an aqueous solution of Genipin at different concentrations. After said cross-linking process, the hydrogels were characterized as described in detail below:

a. Structural analysis through histological techniques.

b. Evaluation of the biomechanical properties.

c. Ex vivo cellular biocompatibility tests.

d. In vivo histological biocompatibility study.

e. Blood analysis.

In the case of in vivo studies, Wistar rats were used; hydrogels with and without chemical cross-linking were implanted in subcutaneous cell tissue at the dorsal level in said rats, and the biocompatibility of the biomaterials a level histological, blood, and serum level was evaluated.

Manufacturing Fibrin-Agarose Hydrogels

The production of the fibrin-agarose (FAH) constructs was performed following protocols described hereinabove. Namely for this example, for producing 30 ml of FAH 2.25 ml of PBS are added to 22.8 ml of human blood plasma from healthy human blood donors supplied according to the existing regulations by the CRTSBT (Centro Regional de Transfusión Sanguinea y Banco de Tejidos de Granada y Almeria (Regional Blood Transfusion and Tissue Bank Center of Granada and Almeria)). Then 450 μl of tranexamic acid (Amchafibrin®, FidesEcofarma, Valencia, Spain) are added for the purpose of preventing spontaneous fibrinolysis of the fibrin. 3 ml of Cl2C at 0.025 mM are added to the previous solution to start the fibrin coagulation reaction, and finally 1.5 ml of type VII agarose at 2% dissolved in PBS, previously heated until reaching its melting point, are added. The resulting product is carefully mixed and kept at 37° C. until completely gelled, after having been distributed in 6-well plates.

Cross-Linking FAH with Genipin

Cross-linking with Genipin (GP) is then performed. To that end, the constructs were immersed in 5 ml of each solution of Genipin at 0.1 and 0.25% for 72 h at 37° C. and then rinsed several times with PBS over the span of 24 h.

Four experimental groups are thereby established: control group, FAH, FAH-0.1% GP and FAH-0.25% GP, which were evaluated at two times, as indicated below. Each group was evaluated in triplicate (n=3 each).

In Vivo Implantation of FAH and Obtaining Samples

For the in vivo study of these biomaterials, 3×3×1.5 mm implants were obtained from the gels hosted in Wistar rats, the experimental animals used in this experiment. After removing the necessary fur, these implants were carefully inserted in the dorsum of rats through an incision along the axial axis with a scalpel, in both pockets which were created in the subcutaneous cell tissue with blunt-tip scissors on both sides. The macroscopic appearance of the implants can be seen in FIG. 10.

The analysis to be carried out was done at two times, 12 and 26 days after the inclusion of the implant. Said analysis comprises the sample represented by the implant, a portion of adjacent skin and subcutaneous tissue, kidney, liver, lung, and spleen samples, as well as blood samples used to obtain the blood count.

These studies on the rats, from the implantation of the hydrogel until the drawing of samples, were carried out in the Experimental Surgery Unit of Hospital Universitario Virgen de las Nieves under the supervision of qualified personnel.

Processing of Samples and Stains

For analyzing under a light microscope of the area of the implant and of the various organs, these samples were processed as described in detail below. First, they were fixed in a 10% formol solution, and then they were included in paraffin after dehydration in solutions with increasing concentrations of alcohol and using benzene as the intermediate. Paraffin blocks housing the samples are obtained as a final result. Serial histological sections that are 5 μm thick are taken from said blocks in a paraffin microtome and are finally collected on a slide. For viewing under a light microscope, the histological sections were deparaffinized, hydrated, and subsequently stained in either hematoxylin-eosin (samples from the area of the implant and the remaining organs) or else by means of picrosirius staining (only the samples from the area of the implant).

a. Hematoxylin-Eosin Staining:
Harris hematoxylin for six minutes, after deparaffinization and hydration.
Washing under running water for 10 minutes.
Eosin for 3 minutes.
Dehydration in solutions with increasing alcohol concentrations, rinsing in xylol, and mounting with coverslip.

b. Picrosirius staining (Carriel, 2011):
Picrosirius (picric acid-sirius red) for 30 minutes.
Washing in distilled for water 2 minutes.
Hematoxylin for 3 minutes.
Washing under running water for 4 minutes.
Dehydration in solutions with increasing alcohol concentrations, rinsing in xylol and mounting with coverslip.

Macroscopic and Histological Analysis of the Implants, Tissues and Organs

Biocompatibility is one of the most important characteristics that a biomaterial used in tissue engineering must present, and it is defined by the inflammatory reaction it generates in the tissue hosting it. To study this characteristic, the histological evaluation of the tissues adjacent to the implanted biomaterial, which is a conventional method, has been performed (Anderson and Miller, 1984. Biomaterials 5:5-10).

Histological analysis is performed from the macroscopic and microscopic study of the processed samples.

a. Macroscopic study: This is done by means of direct inspection of the portion of skin corresponding to the implant and of the implant itself, taking into consideration possible parameters that may be present such as signs of inflammation, infection, and necrosis, changes in volume and texture of the implant, or migration thereof.

b. Microscopic study: This is done by taking into consideration the presence and type of inflammatory reaction accompanying the implant, as well as the existence of fibrosis and/or the formation of a fibrous capsule. Parameters relating to the implant such as its degree of degradation, presence of cell components of the inflammatory reaction in its thickness and the possible presence therein of elements such as giant cells, granulomas, or neovessels, were also characterized.

Analysis of the Histological Pattern of Kidneys, Liver, Spleen, and Lungs

Similarly to the samples of the implants, this analysis includes a macroscopic evaluation and a microscopic evaluation, considering the presence of any parameter indicating the existence of inflammation, fibrosis, or necrosis.

Results

A rat control group was used in the experiment. As is to be expected, the histological examination of the skin of these rats showed no alteration whatsoever at either of the two times they were evaluated, after 12 and 26 days (FIG. 11).

Fibrin-agarose hydrogel

Macroscopic Study

At the macroscopic level, no sign of inflammation, necrosis, or infection, and no other alteration in the area of skin that covered the implant could be seen at the time of extraction. There was no implant migration, and its volume seems to have subtly decreased after 26 days, without contrasts in texture.

Microscopic Study

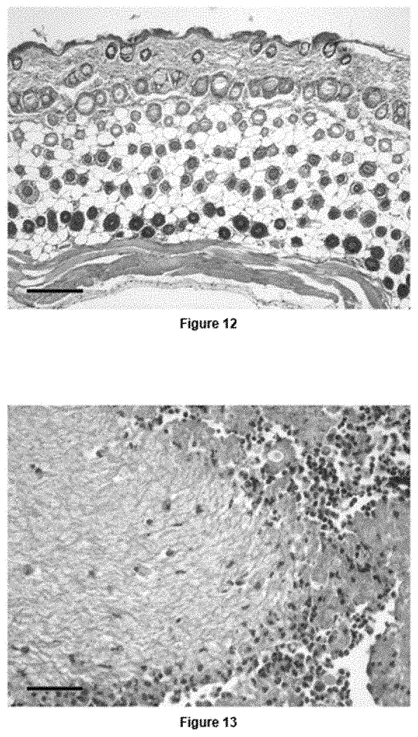

a. After 12 days (FIGS. 12 and 13): there is observed an acute inflammatory reaction of lymphoplasmacytic and neutrophilic predominance around the implant, the degradation of which in the area of the margins already starts to be evident. The presence of macrophages is reduced.

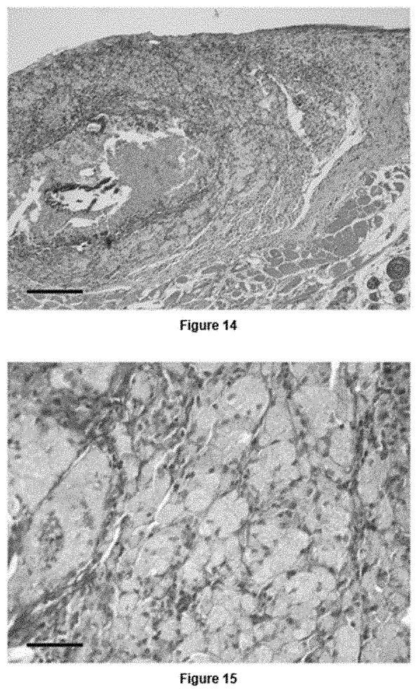

b. After 26 days (FIGS. 14 and 15): degradation of the hydrogel is notable after 26 days, and the inflammation is maintained, although now with considerable histiocytic participation. There does not seem to be a clear formation of a fibrous capsule around the implant, but a considerable increase in fibrosis is observed with picrosirius staining.

At neither of the two times are giant cells or foreign body granulomas observed, nor is they any neovessel formation.

Fibrin-agarose hydrogel after cross-linking with 0.1% Genipin

Macroscopic Study

Macroscopic observation did not point to any alteration of the skin in the area of the implant at either of the two times. There was no remote migration of the implants towards another area of the skin, nor was there any evident change in volume or texture.

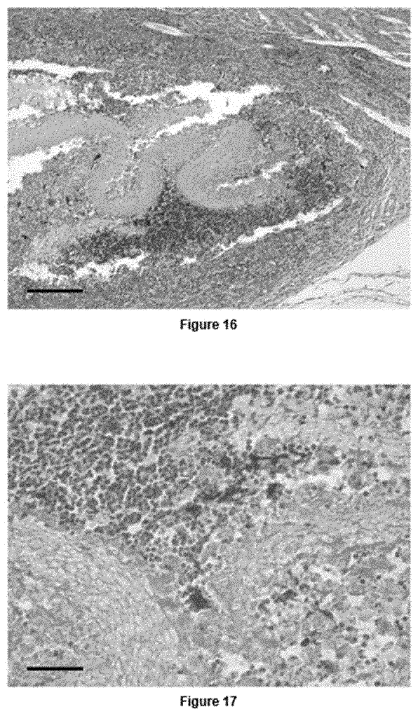

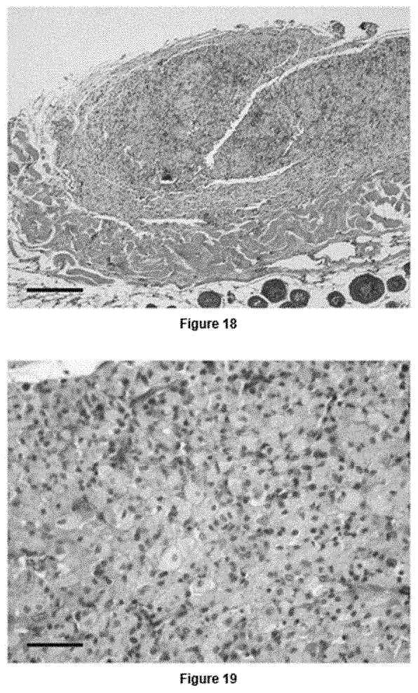

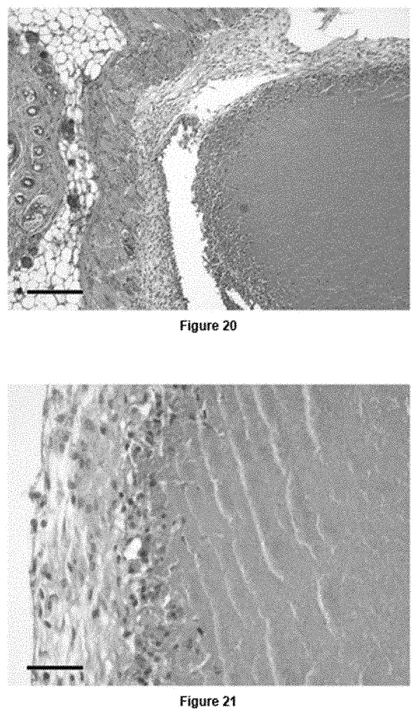

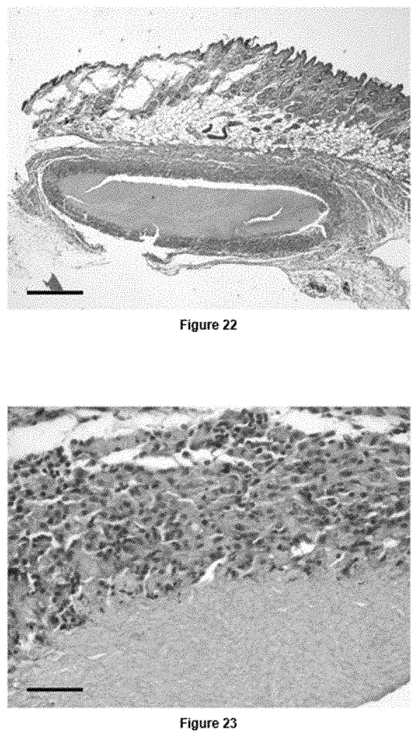

Microscopic Study
After 12 days (FIGS. 16 and 17): there is observed an infiltrate of lymphoplasmacytic and neutrophilic predominance at the margins of the implant that advances towards the interior, with partial degradation of the hydrogel.
After 26 days (FIGS. 18 and 19): degradation of the hydrogel is so evident that there are fewer residues thereof seen in the area of the implant. Conversely, there is observed an infiltrate with a predominance of macrophages where the hydrogel was located. There can likewise be seen an increase in fibrosis, where there is a more or less well demarcated formation of a fibrous capsule around the implant. Conversely, at neither of the two times are giant cells or foreign body granulomas observed, nor is they any neovessel formation.

Fibrin-agarose hydrogel after cross-linking with 0.25% Genipin

Macroscopic Study

The macroscopic study does not show type of alteration in the skin covering the implant of the invention, which did not migrate from its original insertion location either. The implant shows no change whatsoever to the touch, neither in volume nor in texture.

Microscopic Study
After 12 days (FIGS. 11 and 12): the examination under a light microscope shows an infiltrate of lymphoplasmacytic predominance which is only limited to the margins of the hydrogel, with scarce penetration of these elements in the interior of same. There is virtually nil destruction of the hydrogel.
After 26 days (FIGS. 13 and 14): the inflammation continues to be of lymphoplasmacytic predominance, although with a slight increase in the presence of macrophages after 26 days. Nevertheless, said infiltrate is still limited to the margins of the hydrogel with scarce penetration of cells in the interior of same. There is still minimal degradation of the material and it is limited to the exterior. There is no fibrosis in its thickness, although the formation of a fibrous capsule around same can be seen; nor are giant cells, foreign body granulomas, or neovessels observed.

Analysis of the Samples Obtained from Kidneys, Liver, Spleen and Lung.

At the time of extraction, no alteration whatsoever could be observed in any of the mentioned organs at the macroscopic level. Microscopically, no significant differences could be observed between the samples obtained from the control group and the rest of groups, and there was no presence of signs of inflammation, fibrosis, or necrosis.

Blood Analysis: Blood Count

The results of the blood count obtained from the samples of blood of the rats are provided in Table 3.

In summary, based on the results of the preceding it could be said in relation to Genipin that:

a. With respect to the biomechanical characteristics, the rheological analysis shows that the biomechanical patterns represented by the rigidity modulus and the elastic and viscous moduli significantly improve in the gels subjected to cross-linking with Genipin. Among same, the elastic properties of the constructs stand out.

b. With regard to ex vivo cellular biocompatibility, increasing concentrations of Genipin exercise a certain increase in the cytotoxicity of the biomaterial. However, there are significant differences with respect to cell viability that are favorable for 0.25% Genipin with respect to higher concentrations or other agents.

With respect to the biocompatibility of these hydrogels in rat tissues the obtained results indicate the following:
Under macroscopic examination, no migration or notable change in the texture or volume of the implants is observed in any of the hydrogels after the first or second time. Nor are any evident signs of infection, inflammation, or necrosis observed. The examination of the kidneys, liver, spleen, and lung does not show any noticeable alteration or differences with the control group either, a fact which is on the other hand expected, as any change in relation to inflammation in these organs should not appear after only 26 days.

TABLE 3
Parameters of the blood count obtained from blood samples from all the experimental groups.
BLOOD COUNT RESULTS

| | | | | CONTROL | | FAH | | | |
|---|---|---|---|---|---|---|---|---|---|
| | | | | 12 DAYS | 26 DAYS | 12 DAYS | | 26 DAYS | |
| | | | | mean | mean | mean | SD | mean | SD |
| BLOOD COUNT | | WBC | μL⁺¹ | 2.40E+03 | 3.40E+03 | 1.97E+03 | ±9.01E+02 | 3.80E+03 | ±1.01E+03 |
| | | RBC | μL⁺¹ | 8.83E+06 | 8.47E+06 | 8.97E+06 | ±2.54E+05 | 6.93E+06 | ±1.36E+05 |
| | | HGB | g/dL | 14.10 | 13.60 | 13.93 | ±0.32 | 11.77 | ±0.70 |
| | | HCT | % | 45.00 | 42.40 | 45.60 | ±1.28 | 34.67 | ±0.57 |
| | | MCV | fL | 51.00 | 50.10 | 50.73 | ±0.12 | 50.00 | ±0.40 |
| | | MCH | pg | 16.00 | 16.10 | 15.53 | ±0.64 | 16.93 | ±0.71 |
| | | MCHC | g/dL | 31.30 | 32.10 | 30.63 | ±1.34 | 33.93 | ±1.45 |
| | | PLT | μL⁺¹ | 9.60E+04 | 6.27E+05 | 2.13E+05 | ±1.76E+05 | 2.43E+05 | ±2.25E+05 |
| | WBC/White | LYM | % | 66.20 | 87.00 | 66.87 | ±7.22 | 88.37 | ±3.15 |
| | blood cells | MXD | % | 9.20 | 3.30 | 15.27 | ±2.06 | 2.93 | ±1.93 |
| | | NEUT | % | 24.60 | 9.70 | 17.87 | ±5.33 | 8.70 | ±1.23 |
| | | LYM | μL⁺¹ | 1.60E+03 | 3.00E+03 | 1.33E+03 | ±6.03E+02 | 3.37E+03 | ±1.01E+03 |
| | | MXD | μL⁺¹ | 2.00E+02 | 1.00E+02 | 3.00E+02 | ±1.73E+02 | 1.17E+02 | ±7.64E+01 |
| | | NEUT | μL⁺¹ | 6.00E+02 | 3.00E+02 | 3.33E+02 | ±1.53E+02 | 3.33E+02 | ±5.77E+01 |
| | RBC/Red | RDW_SD | fL | 28.50 | 27.60 | 28.30 | ±0.26 | 27.27 | ±0.21 |
| | blood cells | RDW_CV | % | 14.10 | 13.00 | 14.33 | ±0.57 | 12.37 | ±0.31 |
| | PLT/ | PDW | fL | 7.70 | 7.20 | 6.70 | ±0.57 | 9.03 | ±1.80 |
| | Platelets | MPV | fL | 6.10 | 6.00 | 5.90 | ±0.00 | 6.40 | ±0.20 |
| | | P_LCR | % | 5.20 | 5.20 | 3.75 | ±2.47 | 7.33 | ±2.27 |

| | | | | FAH50.1% GP | | | |
|---|---|---|---|---|---|---|---|
| | | | | 12 DAYS | | 26 DAYS | |
| | | | | mean | SD | mean | SD |
| BLOOD COUNT | | WBC | μL⁺¹ | 1.77E+03 | ±7.23E+02 | 5.27E+03 | ±2.03E+03 |
| | | RBC | μL⁺¹ | 8.68E+06 | ±6.93E+04 | 6.57E+06 | ±3.00E+05 |
| | | HGB | g/dL | 13.97 | ±0.12 | 12.20 | ±0.50 |
| | | HCT | % | 44.80 | ±0.17 | 33.03 | ±1.10 |
| | | MCV | fL | 51.60 | ±0.17 | 50.30 | ±0.62 |
| | | MCH | pg | 16.10 | ±0.20 | 18.60 | ±1.51 |
| | | MCHC | g/dL | 31.17 | ±0.31 | 37.00 | ±2.59 |
| | | PLT | μL⁺¹ | 1.31E+05 | ±1.15E+05 | 2.99E+05 | ±1.15E+05 |
| | | LYM | % | 72.83 | ±17.68 | 86.40 | ±2.78 |
| | | MXD | % | 9.27 | ±7.51 | 3.83 | ±1.85 |
| | WBC/White | NEUT | % | 17.90 | ±11.16 | 9.77 | ±0.97 |
| | blood cells | LYM | μL⁺¹ | 1.23E+03 | ±3.06E+02 | 4.50E+03 | ±1.61E+03 |
| | | MXD | μL⁺¹ | 2.17E+02 | ±1.76E+02 | 2.33E+02 | ±1.15E+02 |
| | | NEUT | μL⁺¹ | 3.33E+02 | ±3.21E+02 | 5.33E+02 | ±3.06E+02 |
| | RBC/Red | RDW_SD | fL | 28.43 | ±0.12 | 27.47 | ±0.35 |
| | blood cells | RDW_CV | % | 13.97 | ±0.25 | 12.63 | ±0.21 |
| | PLT/ | PDW | fL | 6.80 | ±+ | 7.37 | ±0.40 |
| | Platelets | MPV | fL | 6.40 | ±+ | 6.23 | ±0.29 |
| | | P_LCR | % | 9.30 | ±+ | 7.67 | ±1.70 |

| | | | | FAH50.25% GP | | | |
|---|---|---|---|---|---|---|---|
| | | | | 12 DAYS | | 26 DAYS | |
| | | | | mean | SD | mean | SD |
| BLOOD COUNT | | WBC | μL⁺¹ | 2.47E+03 | ±3.51E+02 | 4.67E+03 | ±8.14E+02 |
| | | RBC | μL⁺¹ | 7.87E+06 | ±4.90E+05 | 7.97E+06 | ±7.46E+05 |
| | | HGB | g/dL | 12.33 | ±1.04 | 12.97 | ±0.55 |
| | | HCT | % | 40.30 | ±2.81 | 39.87 | ±3.69 |
| | | MCV | fL | 51.17 | ±1.15 | 50.03 | ±0.29 |
| | | MCH | pg | 15.67 | ±0.35 | 16.33 | ±0.85 |
| | | MCHC | g/dL | 30.60 | ±0.70 | 32.63 | ±1.67 |
| | | PLT | μL⁺¹ | 6.53E+04 | ±6.53E+04 | 5.63E+05 | ±3.27E+05 |
| | | LYM | % | 69.55 | ±2.05 | 82.83 | ±3.20 |
| | | MXD | % | 14.00 | ±11.31 | 9.17 | ±5.60 |
| | WBC/White | NEUT | % | 16.45 | ±9.26 | 8.00 | ±2.44 |
| | blood cells | LYM | μL⁺¹ | 1.85E+00 | ±7.07E+01 | 3.83E+03 | ±5.77E+02 |
| | | MXD | μL⁺¹ | 4.00E+02 | ±2.83E+02 | 4.33E+02 | ±3.06E+02 |
| | | NEUT | μL⁺¹ | 4.00E+02 | ±1.41E+02 | 4.00E+02 | ±1.00E+02 |
| | RBC/Red | RDW_SD | fL | 29.43 | ±2.40 | 27.83 | ±0.15 |
| | blood cells | RDW_CV | % | 14.73 | ±2.92 | 13.13 | ±0.15 |
| | PLT/ | PDW | fL | + | ±+ | 6.80 | ±0.00 |
| | Platelets | MPV | fL | + | ±+ | 6.00 | ±0.14 |
| | | P_LCR | % | + | ±+ | 4.70 | ±1.13 |

The inflammation observed in the area of the implant shares general characteristics among the different hydrogels. After the first time, after 12 days, inflammation has a lymphoplasmacytic predominance, with the participation of polymorphonuclear neutrophils and a scarce presence of macrophages. This infiltrate is seen to be somewhat more intense in FAH and FAH GP-0.1%, in which a small number of inflammatory cells penetrate the thickness of the implant. The blood count data, as can be observed in the table presented above, supports the predominance of lymphocytes over the rest of the cell strains. Degradation of the implant after 12 days is very distinct in FAH GP-0.1%, somewhat lower in FAH, and virtually inexistent in FAH GP-0.25%, being limited in any case to the most marginal area. The fact that FAH GP-0.1% presents a more accelerated apparent degradation than FAH without cross-linking seems to contradict what is to be expected. This result could be attributed to the manner in which Genipin modifies the structural configuration of the hydrogel, probably making it easier to be degraded by the immune system at this low concentration.

After 26 days (second time), inflammation presents a distinct increase in macrophages compared with the analysis after 12 days, being much lower in FAH GP-0.25%. This increase in macrophages is accompanied by an advance in the hydrogel degradation process, which is almost complete in the case of FAH and FAH GP-0.1%. Nevertheless, FAH GP-0.25% continues virtually intact, inflammation and degradation again being limited to the outermost area and in masked form. It therefore seems worth highlighting that the structure and biomechanical characteristics of FAH GP-0.25% vary such that their degradation is very significantly delayed compared with the concentration of Genipin at 0.1% or the absence thereof in the hydrogel.

A more evident increase in fibrosis around the implants can also be observed this second time in FAH, FAH GP-0.1% and FAH GP-0.25%, outlining a fibrous capsule in a more or less clear manner. Conversely, there are no phenomena of infection or necrosis, or the presence of giant foreign body cells or granulomas, or the formation of other elements such as neovessels observed in any of the implants at either of the two times.

Taking into account the data obtained after the experiments performed, it seems evident to assert that the variability of characteristics presented ex vivo by the different biomaterials according to their composition likewise represents an in vivo variability of the response of live tissue vivo with respect to same. Knowing that the inflammatory response of the tissue to the implant presents similar characteristics in all the analyzed hydrogels, the principal difference between them in vivo resides in the particularly slow degradation process characterizing FAH GP-0.25%. This variation in degradation can be used for designing implants with a variable degradation rate, as may be desirable in the medical application.

1. A biomaterial comprising:
a) fibrinogen;
b) an antifibrinolytic agent;
c) an element selected from: a coagulation factor, a source of calcium, thrombin, or any combinations thereof;
d) a polysaccharide; and
e) a compound of formula (I) 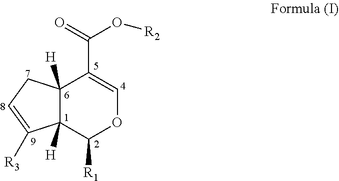
or any of the esters, tautomers, and pharmaceutically acceptable salts thereof, wherein:
R₁ is —H, ═O or —OR₄, wherein R₄ is —H, C₁₋₆ alkyl, C₁₋₃ alkyl, or C₁₋₁₂ alkanoyl which can be substituted with phenyl, phenoxy, pyridyl, or thienyl;
R₂ is H, C₁₋₆ alkyl, C₁₋₃ alkyl, methyl, ethyl, propyl, isopropyl, butyl, w-butyl, i-butyl, isobutyl, or sec-butyl; and
R₃ is a primary alcohol selected from —CH₂—OH and —R₅—CH₂—OH, where —R₅— is C₁₋₆ alkyl, C₁₋₃ alkyl, methyl, ethyl, propyl, isopropyl, butyl, n-butyl, t-butyl, isobutyl, sec-butyl.

2. The biomaterial according to claim, 1 where R₁ is —OR₄

3. The biomaterial according to claim 1, wherein R₄ is —H or C₁₋₃ alkyl

4. The biomaterial according to claim 1, wherein R₂ is H or C₁₋₃ alkyl or R₃ is —CH₂—OH, —CH₂—CH₂—OH, or —CH₂—CH₂—CH₂—OH.

5. The biomaterial according to claim 1, wherein R₂ is H or C₁₋₃ alkyl.

6. The biomaterial according to claim 1, wherein R₃ is —CH₂—OH, —CH₂—CH₂—OH, —CH₂—CH₂—CH₂—OH.

7. The biomaterial according to claim 1, wherein the compound of formula (I) has formula (II):

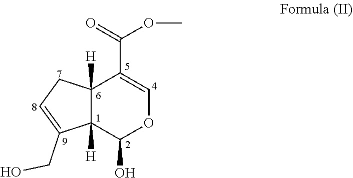 [SMILES: COC(=O)C1=CO[C@@H](O)[C@@H]2C(CO)=CC[C@H]12]

8. The biomaterial according to claim 1, wherein the origin of the fibrinogen or fibrin is blood plasma.

9. The biomaterial according to claim 8, wherein the blood plasma is of autologous origin.

10. The biomaterial according to claim 1, wherein the antifibrinolytic agent is tranexamic acid.

11. The biomaterial according to claim 1, wherein the source of calcium is a calcium salt.

12. The biomaterial according to claim 11, wherein the calcium salt is calcium chloride.

13. The biomaterial according to claim 1, wherein the polysaccharide is agarose.

14. The biomaterial according to claim 13, wherein the agarose is type VII agarose.

15. The biomaterial according to claim 1, further comprising a protein.

16. The biomaterial according to claim 15, wherein the protein is selected from fibronectin, collagen, or the combination thereof.

17. The biomaterial according to claim 15, wherein the protein is type I collagen.

18. (canceled)

19. An artificial tissue comprising the biomaterial according to claim 1, and further comprising mammalian cells.

20-34. (canceled)

35. A method for increasing, restoring, or partially or completely substituting the functional activity of a diseased or damaged tissue or organ in a subject in need thereof, comprising implanting the artificial tissue of claim 19 in the subject.

36. (canceled)

37. The biomaterial of claim 1, wherein R₂ is H or C₁₋₃ alkyl and R₃ is —CH₂—OH, —CH₂—CH₂—OH, or —CH₂—CH₂—CH₂—OH.
